(19) United States
(12) Patent Application Publication
(10) Pub. No.: US 2008/0032989 A1
(43) Pub. Date: Feb. 7, 2008

(54) Method of treating inflammatory diseases using tyroskine kinase inhibitors

(76) Inventors: William H. Robinson, Palo Alto, CA (US); Ricardo T. Paniagua, Redwood City, CA (US)

Correspondence Address: PERKINS COIE LLP, P.O. BOX 2168, MENLO PARK, CA, 94026, US

(21) Appl. No.: 11/809,515

(22) Filed: May 31, 2007

Related U.S. Application Data
(60) Provisional application No. 60/810,030, filed on May 31, 2006.

(51) Int. Cl. A61K 31/496; A61K 31/403; A61K 31/506; A61K 31/519; A61P 17/06; A61P 19/02

(52) U.S. Cl. 514/252.18; 514/264.11; 514/275; 514/414; 514/789

(57) ABSTRACT

Methods for treating and preventing inflammatory diseases using tyrosine kinase inhibitors are described. The inhibitors inhibit, e.g., T lymphocyte and/or B lymphocyte function, fibroblast proliferation, mast cells activation, and/or monocyte differentiation.

### CROSS REFERENCE TO RELATED APPLICATIONS

This application claims the benefit of U.S. Provisional Application No. 60/810,030, filed May 31, 2006, incorporated herein by reference in its entirety.

### STATEMENT REGARDING GOVERNMENT INTEREST

This work was supported in part by NIH K08 AR02133, NIH NHLBI contract N01 HV 28183, a NIH F31 Fellowship Award, and Department of Veterans Affairs funding. Accordingly the United States government may have certain rights in this invention.

### TECHNICAL FIELD

The subject matter described herein relates to a method of treating inflammatory diseases with tyrosine kinase inhibitors.

### BACKGROUND

Inflammatory and autoimmune diseases are estimated to affect 3-5% of the U.S. and world populations (Jacobson et al. (1997) Clin Immunol. Immunopathol. 84:223-43). In normal individuals immune responses provide protection against viral and bacterial infections. In autoimmune diseases, these same cellular responses target host tissues, causing organ and/or tissue damage, e.g., to the joints, skin, pancreas, brain, thyroid or gastrointestinal tract). Further manifestations of autoimmune disorders are caused by dysregulated host cell responses in the chronic inflammatory state.

The methods and compositions to be described relate to tyrosine kinases and inflammatory diseases, disorders, and conditions, for which the following background information is provided.

A. Tyrosine Kinases

Phosphorylation of target proteins by kinases is an important mechanism in signal transduction and for regulating enzyme activity. Tyrosine kinases (TK) are a class of over 100 distinct enzymes that transfer a phosphate group from ATP to a tyrosine residue in a polypeptide (Table 1). Tyrosine kinases phosphorylate signaling, adaptor, enzyme and other polypeptides, causing such polypeptides to transmit signals to activate (or inactive) specific cellular functions and responses. There are two major subtypes of tyrosine kinases, receptor tyrosine kinases and cytoplasmic/non-receptor tyrosine kinases.

1. Receptor Tyrosine Kinases

To date there have been approximately 60 receptor tyrosine kinases (RTK; also known as tyrosine receptor kinases (TRK)) described in humans. These kinases are high affinity receptors for hormones, growth factors and cytokines (Table 1) (Robinson et al. (2001) Oncogene 19:5548-57). The binding of hormones, growth factors and/or cytokines generally activates these kinases to promote cell growth and division. Exemplary insulin-like growth factor receptor, epidermal growth factor receptor, platelet-derived growth factor receptor, etc.). Most receptor tyrosine kinases are single subunit receptors but some, for example the insulin receptor, are multimeric complexes. Each monomer contains an extracellular N-terminal region, a single transmembrane spanning domain of 25-38 amino acids, and a C-terminal intracellular domain. The extracellular N-terminal region is composed of a very large protein domain which binds to extracellular ligands e.g. a particular growth factor or hormone. The C-terminal intracellular region provides the kinase activity of these receptors. To date, approximately 20 different subclasses of receptor tyrosine kinases have been identified (Table 1) (Robinson et al. (2001) Oncogene 19:5548-57). Receptor tyrosine kinases are key regulators of normal cellular processes and play a critical role in the development and progression of many types of cancer (Zwick et al. (2001) Endocr. Relat. Cancer 8:161-173).

RTKs include an extracellular binding site for their ligand, a transmembrane domain, and a kinase domain within the cytoplasm. The RTKs further include an ATP-binding site, a domain to bind the kinase substrate, and a catalytic site to transfer the phosphate group. The catalytic site lies within a cleft which can be in an open (active) or closed (inactive) form. The closed form allows the substrate and other residues to be brought into the catalytic site, and the open form grants access to ATP to drive the catalytic reaction (Roskoski, R. (2005) Biochem. Biophys. Res. Commun. 338:1307-15).

The class III RTKs, which include PDGFRα, PDGFRβ, c-Fms, c-Kit and Fms-like tyrosine kinase 3 (Flt-3), are distinguished from other classes of RTKs in having five immunoglobulin-like domains within their extracellular binding site as well as a 70-100 amino acid insert within the kinase domain (Roskoski, R. (2005) Biochem. Biophys. Res. Commun. 338:1307-15). Structural similarities among class III RTKs results in cross-reactivity with respect to ligands, as evidenced in the case of imatinib blocking PDGFRa, PDGFRb, c-Fms, and c-Kit. Platelet-derived growth factor receptors (PDGFR) include PDGFR-alpha (PDGFRα) and the PDGFR-beta (PDGFRβ) (Yu, J. et al, (2001) Biochem Biophys Res Commun. 282:697-700). The PDGF B-chain homodimer PDGF BB activates both PDGFRα and PDGFRβ, and promotes proliferation, migration and other cellular functions in fibroblast, smooth muscle and other cells. The PDGF-A chain homodimer PDGF AA activates PDGFRα only. PDGF-AB binds PDGFRα with high-affinity and in the absence of PDGFRα can bind at a lower affinity (Seifert, R. A. et al. (1993) J. Biol. Chem. 268:4473-80). Recently, additional PDGFR ligands have been identified including PDGF-CC and PDGF-DD. Fibroblasts and other mesenchymal cells express fibroblast-growth factor receptor (FGFR) which mediates tissue repair, wound healing, angiogenesis and other cellular functions.

There are several direct and indirect ways to block tyrosine kinase activity, including: (i) competitive inhibition of ATP binding site, (ii) interfering with the cleft transition from open to closed forms (i.e., stabilizing either the open or closed forms), (iii) directly blocking the substrate from binding to the binding site of a tyrosine kinases, and (iv) blocking production or recruitment of ligand or substrate. Imatinib, CGP53716 and GW2580 are examples of small molecule tyrosine kinase inhibitors that are competitive inhibitors of ATP binding to the kinase. Imatinib binds the closed (inactive) form of Abl, while the open (active) form is sterically incompatible for imatinib binding. ATP cannot bind to the TK when imatinib is bound, and the substrate cannot be phosphorylated. The small molecule tyrosine kinase inhibitors approved to date (Table 2) bind the ATP-binding site and block ATP from binding, thereby inhibiting the tyrosine kinase from phosphorylating its substrate target.

TABLE 1
Tyrosine Kinases: Overview of Cellular Distributions and Cellular Functions

| | | |
|---|---|---|
| Tyrosine kinase | Cells expressing kinase | Cellular function |

Receptor:

| | | |
|---|---|---|
| PDGFR family: | | |
| c-Fms | Monocytes, macrophages, osteoclasts | Cell growth, proliferation, differentiation, survival, and |
| | | priming |
| PDGFRα | Fibroblasts, smooth muscle cells, keratinocytes, | Cell growth, proliferation, differentiation and survival |
| | glial cells, chondrocytes | |
| PDGFRβ | Fibroblasts, smooth muscle cells, keratinocytes, | Cell growth, proliferation, differentiation and survival |
| | glial cells, chondrocytes | |
| c-Kit | Haematopoietic progenitor cells, mast cells, | Cell growth, proliferation, differentiation and survival |
| | primordial germ cells, interstitial cells of Cajal | |
| Flt-3 | Haematopoietic progenitor cells | Cell growth, proliferation, differentiation and survival |
| VEGFR family: | | |
| VEGFR1 | Monocytes, macrophages, endothelial cells | Monocyte and macrophage migration; vascular |
| | | permeability |
| VEGFR2 | Endothelial cells | Vasculogenesis; angiogenesis |
| VEGFR3 | Lymphatic endothelial cells | Vasculogenesis; lymphangiogenesis |
| FGFR family: | Fibroblasts and other mesenchymal cells | Tissue repair, wound healing, angiogenesis |

Non-receptor (cytoplasmic):

| | | |
|---|---|---|
| ABL family: | Ubiquitous | Cell proliferation, survival, cell adhesion and migration |
| JAK family: | | |
| JAK1 | Ubiquitous | Cytokine signaling |
| JAK2 | Ubiquitous | Hormone-like cytokine signaling |
| JAK3 | T cells, B cells, NK cells, myeloid cells | common-gamma chain cytokine signaling |
| TYK2 | Ubiquitous | Cytokine signaling |
| SRC-A family: | | |
| FGR | Myeloid cells (monocytes, macrophages, | Terminal differentiation |
| | granulocytes) | |
| FYN | Ubiquitous | Cell growth; T cell receptor, regulation of brain function, |
| | | and adhesion mediated signaling |
| SRC | Ubiquitous | Cell development, growth, replication, adhesion, |
| | | motility |
| YES | Ubiquitous | Maintaining tight junctions; transmigration of IgA across |
| | | epithelial cells |
| SRC-B family: | | |
| BLK | B cells, thymocytes | B cell proliferation and differentiation, thymopoiesis |
| HCK | Myeloid cells, lymphoid cells | Proliferation, differentiation, migration |
| LCK | T cells, NK cells | T-cell activation, KIR activation |
| LYN | Myeloid cells, B cells, mast cells | BCR signaling; FceR1 signaling |
| SYK family: | | |
| SYK | Ubiquitous | Proliferation, differentiation, phagocytosis; tumor |
| | | suppressor |
| ZAP70 | T cells, NK cells | T-cell activation; KIR activation |

2. Cytoplasmic/Non-Receptor Tyrosine Kinases

Over 30 cytoplasmic tyrosine kinases have been described in humans (Table 1). The first cytoplasmic tyrosine kinase identified was viral Src (v-Src), which represents a mutated, constitutively active form of mammalian Src that can transform normal cells into cancer cells. SRC family members have been found to regulate many cellular processes. For example, the T-cell antigen receptor leads to intracellular signaling by activation of Lck and Fyn, two proteins that are structurally similar to Src. Abl (or c-Abl) is a member of the ABL family of non-receptor tyrosine kinases, and mediates cell proliferation, survival, adhesion and migration. The Bcr-Abl chromosomal translocation (the Philadelphia chromosome) causes overexpression of Abl, which results in uncontrolled cell growth and the development of chronic myelogenous leukemia (CML).

B. Development of Small Molecule Kinase Inhibitors to Treat Cancer.

Small molecule tyrosine kinases inhibitors have been developed for the treatment of cancer. For example, imatinib mesylate (GLEEVEC) was developed to inhibit Abl for treatment of chronic myelogenous leukemias associated with the Philadelphia chromosome Bcr-Abl translocation. Small molecule tyrosine kinase inhibitors for the treatment of cancer are listed in Table 2.

TABLE 2
FDA-Approved Tyrosine Kinase Inhibitors and Their Clinical Indications

| Compound | Tradename | Company | Approval date | FDA-Approved Uses |
|---|---|---|---|---|
| Imatinib | GLEEVEC | Novartis | May 2001 | Chronic myeloid leukemia, gastrointestinal |
| | | | | stromal tumors |
| Gefitinib | IRESSA | AstraZeneca | May 2003 | Non-small cell lung cancer |
| Erlotinib | TARCEVA | OSI Pharms | November 2004 | Non-small cell lung cancer |
| Sorafenib | NEXAVAR | Bayer | December 2005 | Advanced renal cell carcinoma |
| Sunitinib | SUTENT | Pfizer | January 2006 | Gastrointestinal stromal tumors, advanced |
| | | | | renal cell carcinoma |
| Dasatinib | SPRYCEL | Bristol Myers | June 2006 | Chronic myeloid leukemia, Ph−+ acute |
| | | | | lymphoblastic leukemia |
| Lapatinib | TYKERB | GlaxoSmithKline | March 2007 | HER2-positive breast cancer |

A major objective of the pharmaceutical and biotechnology industry is development of drugs that are highly specific for a desired target protein or cell, to minimize side effects and toxicities due to “off-target” effects. The cancers described in Table 2 are mediated by genetic mutations in kinases (or mutations resulting in overexpression of kinase genes), and for the treatment of such cancers it is most desirable to utilize a highly specific tyrosine kinase inhibitor to maximize efficacy relative to toxicity. As a result, significant efforts have been undertaken to identify compounds that only bind a single TK.

Imatinib was developed and approved by the FDA to inhibit Abl (in the case of the Bcr-Abl translocation genotype). Imatinib was subsequently found to also inhibit Kit, and is approved by the FDA for the treatment of Kit-expressing gastrointestinal stromal tumors (GIST). More recently, it was observed that imatinib also inhibits Fms and PDGFR. A recent study of patients with CML who were treated with 400 mg per day imatinib over 5 years showed that >40% of patients experienced edema, nausea, muscle cramps, musculoskeletal pain, and rashes (Druker B J et al. (2006) N. Engl. J. Med. 355:2408-17). A significant percentage of imatinib-treated patients also developed bone marrow suppression, with 17% of patients exhibiting neutropenia, 9% thrombocytopenia and 4% anemia (Druker B J et al. (2006) N Engl J Med 355: 2408-2417). Cardiotoxicity has also been described in imatinib-treated patients, and might be due to mitochondrial and sarcoplasmic reticulum dysfunction secondary to inhibition of Abl (Kerkela R et al. (2006) Nat. Med. 12:908-16).

Subtle structural differences in the ATP-binding sites of TKs allow some specificity for small molecule inhibitors for certain tyrosine kinases but not others. In the example of imatinib blocking Abl activity, imatinib makes extensive contacts with peptide segments both within the cleft and outside of the cleft (Hubbard, S. (2002) Curr. Opin. Struct. Biol. 12:735-41).

C. Autoimmune Diseases

Rheumatoid arthritis: In rheumatoid arthritis (RA) the synovial (lined) joints are attacked by the adaptive immune system and patients exhibit arthritis. Aberrant host immune and tissue cell responses play a central role in pathogenesis, with chronic autoimmune inflammation resulting in the trafficking of large number of neurotrophils, macrophage and lymphocytes into the synovium which results in: (i) activation of these infiltrating immune cells to produce pro-inflammatory cytokines including TNFα and IL-6, (ii) production, release and activation of degraditive enzymes including matrix metalloproteinases (MMPs) and other enzymes that breakdown and destroy joint tissues, (iii) inflammation-induced proliferation and hyperplastic growth of the synovial lining to invade and destroy adjacent joint tissues. Although the etiology of rheumatoid arthritis remains unknown, macrophage, neutrophils, mast cells, T and B cells, and fibroblast-like synoviocyte (FLS) become activated in and contribute to synovial inflammation and joint destruction.

In RA, monocytes differentiate into macrophages that infiltrate the synovium and secrete TNFα and other proinflammatory cytokines that potentiate inflammation (Burmester, G. R. et al. (1997) Arthritis Rheum 40:5-18; Kinne, R. W. et al. (2000) Arthritis Res 2:189-202) and osteoclasts that erode bone. TNFα plays a central role in synovitis and joint destruction in murine arthritis (Kontoyiannis, D. et al. (1999) Immunity 10:387-398) and human rheumatoid arthritis (Weinblatt, M. E. et al. (1999) N. Engl. J. Med. 340:253-259).

Multiple sclerosis: Multiple sclerosis (MS) is a debilitating, inflammatory, neurological illness characterized by demyelination of the central nervous system. The disease primarily affects young adults with a higher incidence in females. Symptoms of the disease include fatigue, numbness, tremor, tingling, dysesthesias, visual disturbances, dizziness, cognitive impairment, urological dysfunction, decreased mobility, and depression. Four types classify the clinical patterns of the disease: relapsing-remitting, secondary progressive, primary-progressive and progressive-relapsing (S. L. Hauser and D. E. Goodkin, Multiple Sclerosis and Other Demyelinating Diseases in Harrison's Principles of Internal Medicine 14th Edition, vol. 2, McGraw-Hill, 1998, pp. 2409-19).

Systemic sclerosis: Systemic sclerosis (SSc, or scleroderma) is an autoimmune disease characterized by fibrosis of the skin and internal organs and widespread vasculopathy. Patients with SSc are classified according to the extent of cutaneous sclerosis: patients with limited SSc have skin thickening of the face, neck, and distal extremities, while those with diffuse SSc have involvement of the trunk, abdomen, and proximal extremities as well. Internal organ involvement tends to occur earlier in the course of disease in patients with diffuse compared with limited disease (Laing et al. (1997) Arthritis. Rheum. 40:734-42). The majority of patients with diffuse SSc who develop severe internal organ involvement will do so within the first three years after diagnosis at the same time the skin becomes progressively fibrotic (Steen and Medsger (2000) Arthritis Rheum. 43:2437-44.). Common manifestations of diffuse SSc that are responsible for substantial morbidity and mortality include interstitial lung disease (ILD), Raynaud's phenomenon and digital ulcerations, pulmonary arterial hypertension (PAH) (Trad et al. (2006) Arthritis. Rheum. 54:184-91.), musculoskeletal symptoms, and heart and kidney involvement (Ostojic and Damjanov (2006) Clin. Rheumatol. 25:453-7). Current therapies focus on treating specific symptoms, but disease-modifying agents targeting the underlying pathogenesis are lacking.

Psoriasis: Psoriasis is a chronic skin disease, characterized by scaling and inflammation. Psoriasis affects 1.5 to 2 percent of the United States population, or almost 5 million people. It occurs in all age groups and about equally in men and women. People with psoriasis suffer discomfort, restricted motion of joints, and emotional distress. When psoriasis develops, patches of skin thicken, redden, and become covered with silvery scales, referred to as plaques. Psoriasis most often occurs on the elbows, knees, scalp, lower back, face, palms, and soles of the feet. The disease also may affect the fingernails, toenails, and the soft tissues inside the mouth and genitalia. About 10 percent of people with psoriasis have joint inflammation that produces symptoms of arthritis.

When skin is wounded, a wound healing program is triggered, also known as regenerative maturation. Lesional psoriasis is characterized by cell growth in this alternate growth program. In many ways, psoriatic skin is similar to skin healing from a wound or reacting to a stimulus such as infection, where the keratinocytes switch from the normal growth program to regenerative maturation. Cells are created and pushed to the surface in as little as 2-4 days, and the skin cannot shed the cells fast enough. The excessive skin cells build up and form elevated, scaly lesions. The white scale (called “plaque”) that usually covers the lesion is composed of dead skin cells, and the redness of the lesion is caused by increased blood supply to the area of rapidly dividing skin cells.

The exact cause of psoriasis in humans is not known, although it is generally accepted that it has a genetic component, and a recent study has established that it has an autoimmune component. Whether a person actually develops psoriasis is hypothesized to depend on something “triggering” its appearance. Examples of potential “trigger factors” include systemic infections, injury to the skin (the Koebner phenomenon), vaccinations, certain medications, and intramuscular injections or oral steroid medications. The chronic skin inflammation of psoriasis is associated with hyperplastic epidermal keratinocytes and infiltrating mononuclear cells, including CD4+ memory T cells, neutrophils and macrophages. Macrophage that produce TNF likely play an important role in driving inflammation and pathogenesis in psoriasis.

Systemic lupus erythematosus (SLE): SLE is an autoimmune disease characterized by polyclonal B cell activation, which results in a variety of anti-protein and non-protein autoantibodies (see, e.g., Kotzin et al. (1996) Cell 185:303-06. for a review of the disease). These autoantibodies form immune complexes that deposit in multiple organ systems, causing tissue damage. SLE has a variable course characterized by exacerbations and remissions and is difficult to study. For example, some patients may demonstrate predominantly skin rash and joint pain, show spontaneous remissions, and require little medication. The other end of the spectrum includes patients who demonstrate severe and progressive kidney involvement (glomerulonephritis and cerebritis) that requires therapy with high doses of steroids and cytotoxic drugs such as cyclophosphamide.

Inflammatory bowel diseases: Inflammatory bowel diseases, include Crohn's disease and ulcerative colitis, involve autoimmune attack of the bowel. These diseases cause chronic diarrhea, frequently bloody, as well as symptoms of colonic dysfunction.

Autoimmune diabetes: In autoimmune diabetes, also known as insulin-dependent diabetes mellitus and type I diabetes, the immune system attacks and destroys the beta cells of the pancreas. The beta cells produce insulin, and as a result the afflicted patient becomes insulin deficient and manifests clinical symptoms of diabetes including polyuria, polydypsia and polyphagia. Patients with autoimmune diabetes are treated with insulin injections, and cannot survive without the administration of insulin.

Additional examples of autoimmune diseases include those involving the thyroid (Grave's disease and Hashimoto's thyroiditis), peripheral nerves (Guillain-Barre Syndrome and other autoimmune peripheral neuropathies), the CNS (acute disseminated encephalomyelitis, ADEM), the skin (pemphigoid (bullous), pemphigus foliaceus, pemphigus vulgaris, coeliac sprue-dermatitis, vitiligo), the liver and gastrointestinal system (primary biliary cirrhosis, pernicious anemia, autoimmune hepatitis), and the eye (autoimmune uveitis). There are also multiple “autoimmune rheumatic diseases” (Sjögren's syndrome, discoid lupus, antiphospholipid syndrome, CREST, mixed connective tissue disease (MCTD), polymyositis and dermatomyositis, and Wegener's granulomatosus).

Compounds that modulate immune and host cell responses can be useful in treating these and other diseases associated with inflammation. The present compositions and methods provide a novel approach to treat autoimmune and inflammatory diseases using tyrosine kinase inhibitors.

### BRIEF SUMMARY

The following aspects and embodiments thereof described and illustrated below are meant to be exemplary and illustrative, not limiting in scope.

In one aspect, a method for treating an inflammatory disease is provided, comprising:

orally administering a tyrosine kinase inhibitor to a subject suffering from an inflammatory disease in an amount sufficient to inhibit the activity of at least one tyrosine kinase.

In some embodiments, the tyrosine kinase inhibitor is selected from imatinib, CGP53716, SU9518, PD166326, and GW2580.

In some embodiments, the tyrosine kinase inhibitor is imatinib and the tyrosine kinase is selected from c-Fms, c-Kit, PDGFRα, PDGFRβ, and Abl.

In some embodiments, the tyrosine kinase inhibitor is CGP53716 and the tyrosine kinase is selected from PDGFR (PDGFRα and PDGFRβ), FGFR and c-Kit.

In some embodiments, the tyrosine kinase inhibitor is GW2580 and the tyrosine kinase is selected from c-Fms and PDGFR.

In some embodiments, the tyrosine kinase inhibitor is PD166326 and the tyrosine kinase is selected from c-Kit and Abl.

In some embodiments, the tyrosine kinase inhibitor is SU9518 and the tyrosine kinase is PDGFR and FGFR.

In some embodiments, the inflammatory disease is an autoimmune disease. In particular embodiments, the inflammatory disease is rheumatoid arthritis. In other particular embodiments, the inflammatory disease is systemic sclerosis. In other particular embodiments, the inflammatory disease is multiple sclerosis. In still other particular embodiments, the inflammatory disease is selected from, psoriasis, psoriatic arthritis, Crohn's disease, systemic lupus erythematosus, and pulmonary fibrosis.

In some embodiments, the tyrosine kinase inhibitor is orally administered at a dose that achieves blood levels of about 0.2 micromolar. In some embodiments, the tyrosine kinase inhibitor is orally administered at a dose that achieves blood levels of about 1 micromolar. In some embodiments, the tyrosine kinase inhibitor is orally administered at a dose that achieves blood levels of about 5 micromolar. In some embodiments, the tyrosine kinase inhibitor is orally administered about once per day.

In another aspect, a method for treating an inflammatory disease is provided, comprising orally administering a tyrosine kinase inhibitor to a subject suffering from an inflammatory disease in an amount sufficient to inhibit two or more kinases to treat the inflammatory disease.

In some embodiments, the tyrosine kinase inhibitor is a single compound.

In some embodiments, the tyrosine kinase inhibitor inhibits PDGFR. In some embodiments, the tyrosine kinase inhibitor inhibits c-Kit. In some embodiments, the tyrosine kinase inhibitor inhibits c-Fms. In some embodiments, the tyrosine kinase inhibitor inhibits c-Abl (Abl).

In some embodiments, the tyrosine kinase inhibitor is a single compound that inhibits PDGFR and c-Fms. In some embodiments, the tyrosine kinase inhibitor is a single compound that inhibits PDGFR and c-Abl. In some embodiments, the tyrosine kinase inhibitor is a single compound that inhibits PDGFR and c-Kit. In some embodiments, the tyrosine kinase inhibitor is a single compound that inhibits c-Fms and c-Abl. In some embodiments, the tyrosine kinase inhibitor is a single compound that inhibits c-Fms and c-Kit. In some embodiments the tyrosine kinase inhibitor is a single compound that inhibits FGFR and PDGFR.

In some embodiments, the inflammatory disease is an autoimmune disease.

In particular embodiments, the inflammatory disease is rheumatoid arthritis. In other particular embodiments, the inflammatory disease is systemic sclerosis. In some particular embodiments, the inflammatory disease is multiple sclerosis.

In yet other particular embodiments, the inflammatory disease is selected from, psoriasis, psoriatic arthritis, Crohn's disease, systemic lupus erythematosus, and pulmonary fibrosis.

In some embodiments, the tyrosine kinase inhibitor is orally administered at a dose that achieves blood levels of about 0.2 micromolar. In some embodiments, the tyrosine kinase inhibitor is orally administered at a dose that achieves blood levels of about 1 micromolar. In some embodiments, the tyrosine kinase inhibitor is orally administered at a dose that achieves blood concentrations of about 5 micromolar. In particular embodiments, the tyrosine kinase inhibitor is orally administered about once per day.

In some embodiments, the tyrosine kinase inhibitor is selected from imatinib, CGP53716, SU9518, PD166326, and GW2580.

In addition to the exemplary aspects and embodiments described above, further aspects and embodiments will become apparent by reference to the drawings and by study of the following descriptions.

### BRIEF DESCRIPTION OF THE DRAWINGS

FIGS. 1A-1D are graphs illustrating prevention of collagen induced (CIA) arthritis in mice by treatment with the tyrosine kinase inhibitor, imatinib, at doses of 33 mg/kg (squares) or 100 mg/kg (diamonds), given orally twice-daily starting one day prior to induction of CIA. Control mice were treated with phosphate buffered saline (PBS, circles). FIG. 1A shows the mean arthritis score, assessed using a visual arthritis scoring system in the days following primary immunization. FIG. 1B shows the paw thickness, in mm, in the days following primary immunization. FIG. 1C shows the incidence of arthritis at the termination of the study. FIG. 1D shows the mean weights of mice in each group.

FIGS. 2A and 2B are graphs illustrating treatment of arthritis (average visual arthritis score of 4) in mice having established CIA treated with the tyrosine kinase inhibitor imatinib, at doses of 33 mg/kg (squares) or 100 mg/kg (diamonds) given orally twice-daily. Control mice were treated with phosphate buffered saline (PBS, circles). FIG. 2A shows the mean arthritis score, assessed using a visual arthritis scoring system, in the days following primary immunization. FIG. 2B shows the paw thickness, in mm, in the days following primary immunization.

FIGS. 3A-3I show the results of experiments demonstrating that imatinib reduces synovitis, pannus formation, and joint erosions in CIA. FIGS. 3A-3C are representative H&E stained joint-tissue sections from mice treated in the study described in FIGS. 1A-1D. FIGS. 3D-31 are bar graphs showing histological scores of inflammation, pannus, and bone and cartilage erosions in mice induced for CIA in the prevention (FIGS. 3D-3F) and treatment (FIGS. 3G-31) studies of FIGS. 1A-1D and FIGS. 2A-B, respectively.

FIG. 4A is a photomicrograph of representative joint section from a mouse with CIA, the joint section stained with toluidine blue. Mast cells present in the densely inflamed CIA synovial tissue, as are indicated by arrows. B=bone, JS=joint space. Original magnification 200×.

FIGS. 4B-4D are bar graphs showing that imatinib inhibits mast cell c-Kit activation and pro-inflammatory cytokine production. FIGS. 4B-4D show the concentration of TNFα (FIG. 4B), GM-CSF (FIG. 4C), and IL-6 (FIG. 4D) in mast cells after stimulation for 48 hours with stem cell factor in the presence of 1 μM and 5 μM imatinib.

FIGS. 4E-4F are immunoblots of lysates generated from serum-starved mast cells, pre-incubated with imatinib, and stimulated with stem cell factor for 10 minutes in the presence or absence of imatinib. The immunoblots were probed with antibodies specific for phospho (p)-c-Kit and total c-Kit (FIG. 4E), and p-Akt (Ser 473) and total Akt (FIG. 4F).

FIG. 4G is a reverse phase protein (RPP) array of mast cell lysates generated using the stimulation conditions described for FIGS. 4E-4F. The RPP arrays were probed with a variety of antibodies specific for phosphorylated (activated) protein tyrosine kinases, and the signal levels were normalized to those in unstimulated cells. Yellow represents anti-protein tyrosine kinase antibody reactivity, and blue represents lack of reactivity.

FIGS. 5A-5C show the results of experiments demonstrating inhibition of macrophage c-Fms and downsteam MAPK pathways by imatinib. FIGS. 5A-5B are immunoblots from lysates prepared from isolated resident peritoneal macrophages, serum starved and preincubated with imatinib, and then stimulated with M-CSF for 10 minutes in the presence of imatinib. The immunoblots were probed with antibodies specific for p-c-Fms and total Fms (FIG. 5A) or p-Akt (Ser 473) and total Akt (FIG. 5B). FIG. 5C is an RPP array of peritoneal macrophage lysates generated using the same stimulation conditions. The RPP arrays were probed with a variety of antibodies specific for MAPK pathway and other protein tyrosine kinases, and normalized kinase levels displayed as a heatmap. FIGS. 5D-5F are images of cells showing that imatinib inhibits monocyte differentiation to macrophages. FIG. 5D shows synovial fluid monocytes that were cultured for 72 hours untreated and display the classical round morphology of monocytes. FIG. 5E shows synovial fluid monocytes that were cultured for 72 hours with 100 ng/ml M-CSF. Cells in FIG. 5E clearly display the morphology of macrophages, including multipolar process extension, heterogeneous cytoplasmic vacuoles and inclusions. FIG. 5F shows that imatinib blocks M-CSF-induced differentiation of monocytes to macrophages.

FIGS. 6A-6E show the results of experiments demonstrating that imatinib inhibits B cell functions and epitope spreading. FIG. 6A is a three-dimensional graph showing that B cells were isolated from naïve DBA/1 mouse spleens by negative selection with MACS beads (Miltenyi Biotec) and highly purified by flow cytometry. Isolated B cells were stimulated for 72 hours with 50 μg/ml of μ-specific anti-IgM(Fab′)₂ or 5 ng/ml LPS. FIGS. 6B and 6C are bar graphs showing that imatinib blocks anti-IgM- or LPS-stimulated B cell proliferation. FIG. 6D is a bar graph showing that imatinib also blocks LPS-stimulated B cell production of IgM. FIG. 6E shows a synovial RPP array profile of serum autoantibodies derived from mice with CIA treated with saline or 100 mg/kg imatinib. Antibody reactivity is represented as a heatmap, with the samples from imatinib-treated mice clustering on the right side and demonstrating reduced antibody reactivity against multiple joint antigens.

FIGS. 7A-7E are graphs showing that imatinib inhibits T cell responses. FIG. 7A is an X-Y graph showing the incorporation of ³H-thymidine, as a measure of the proliferation of CII-specific T cells, in splenocytes derived from a mouse expressing a transgene encoding a CII-specific TCR and stimulated with 0-40 μg/mL heat-denatured whole CII in the presence of 0-10 μM imatinib. FIGS. 7B-7E are bar graphs showing the concentration of the cytokines interferon-γ (FIG. 7B), IL-4 (FIG. 7C), TNFα (FIG. 7D) and IL-2 (FIG. 7E) in supernatants of anti-CII TCR transgenic splenocytes stimulated with 20 μg/mL CII from FIG. 7A. Flow cytometry analysis of imatinib-treated cells stained with propidium iodide and Annexin V to determine early apoptosis (PI⁻ Annexin V⁻) as well as late apoptosis or cell death (PI⁺ Annexin V⁻) demonstrated that imatinib did not induce apoptosis or death in these cells.

FIGS. 8A-8E show the results of experiments demonstrating inhibition of cytokine production and fibroblast PDGFR in RA explants. FIGS. 8A-8C are bar graphs showing the cytokine concentrations, TNFα (FIG. 8A), IL-12(p40) (FIG. 8B), and IL-1α (FIG. 8C), in synovial fluid mononuclear cells derived from a human RA patient and stimulated with 100 ng/mL LPS for 48 hrs in the presence of 0-8 μM imatinib. FIGS. 8D-8E are immunoblots showing that imatinib inhibits PDGFR activation in fibroblast-like synoviocytes derived from a human RA patient. Cultured fibroblast-like synoviocytes were preincubated with imatinib followed by stimulation with 25 ng/mL PDGF-BB for 10 minutes. Lysates were produced and probed with antibodies specific for p-PDGFRβ and total PDGFRβ (FIG. 8D) and p-Akt and total Akt (FIG. 8E).

FIGS. 9A-9C are graphs showing that imatinib inhibits fibroblast proliferation in response to PDGF-BB and TGF-β. FIG. 9A is a bar graph showing PDGF-BB-induced proliferation, as measured by ³H-thymidine incorporation, of fibroblast-like synoviocytes (FLS) derived from a human rheumatoid arthritis patient in the presence of 0-6 μM imatinib. FIG. 9B is a graph showing an absence of contaminating macrophage in FLS cell cultures, by flow cytometric analysis with anti-CD68 antibody (FIG. 9B, peritoneal cells=peritoneal macrophage for which CD68 is a lineage marker). FIG. 9C is a bar graph showing that imatinib blocks TGF-β-driven fibroblast proliferation. NIH-3T3 fibroblasts were stimulated with 10 ng/ml TGF-β in the presence of 0-2 μM imatinib. There was robust proliferation following TGF-β stimulation, and proliferation was significantly reduced back to the levels of unstimulated cells with the addition of 0.15 μM or higher concentrations of imatinib.

FIG. 10A is a graph showing that imatinib delays the onset and reduces the severity of murine EAE (a model for multiple sclerosis). The mean EAE score for control (saline-treated) and imatinib (100 mg/kg)-treated EAE mice is shown in the days following EAE induction.

FIGS. 11A and 11B are graphs shown that IC₅₀s of CGP53716, imatinib (Gleevec), and GW2580, for the tyrosine kinases c-Kit and PDGFR. FIG. 11A is a graph showing results from an in vitro c-Kit phophosphorylation assay to determine the IC₅₀ of CGP53716 (0.002 μM), imatinib (0.09 μM) and GW2580 (74 μM) for c-Kit. Recombinant c-Kit was pre-incubated with the indicated small molecule inhibitors, ATP and substrate were added to initiate the phosphorylation reaction, the reactions stopped after 30 minutes, anti-phospho-tyrosine staining performed to detect phospho-c-Kit, and time-resolved fluorescence used to quantitate c-Kit phosphorylation (HTScan c-Kit Kinase Assay, Cell Signaling). FIG. 11A demonstrates that CGP53716 and imatinib both potently inhibit c-Kit, and that GW2580 does not inhibit c-Kit at a clinically relevant concentration (below 5 μM). FIG. 11B shows the results of an in vitro fibroblast-like synoviocyte (FLS) proliferation assay to determine the IC₅₀ of CGP53716 (0.011 μM), imatinib (0.147 μM) and GW2580 (4.35 μM) for PDGFbb-induced proliferation. FLS derived from a human RA patient (at passage 4-6) were pre-incubated with a range of concentrations of the specific inhibitors, stimulated with PDGFbb (which binds both PDGFRα and PDGFRβ), and proliferation measured by H³-thymidine incorporation. FIG. 11B demonstrates that CGP53716, imatinib and GW2580 all inhibit PDGFR at plasma concentrations achieved by standard murine dosing regimens. The IC₅₀ is the concentration of the small molecule kinase inhibitor at which 50% of kinase activity (FIG. 11A) or its corresponding cellular response (FIG. 11B) is inhibited.

FIGS. 12A-12E show the results of experiments demonstrating that the Fms and PDGFR inhibitor GW2580 treats established CIA in a rodent model for rheumatoid arthritis. FIGS. 12A and 12B are graphs showing that treating CIA mice for 5 days with 50 mg/kg reduced the severity of arthritis as compared to vehicle-treated control mice, based on mean visual arthritis scores (FIG. 12A) and paw thickness measurements (FIG. 12B). Error bars represent standard error of the mean (A, B), and asterisks indicate p<0.05 by Mann Whitney statistics (B). FIG. 12C is a schematic of monocyte lineage cell differentiation and functions showing various points in the pathways that a small molecule inhibitor of c-Fms such as GW2580 or imatinib may function. In FIG. 12D-12E, monocytes were isolated from human rheumatoid arthritis patient and stimulated with M-CSF (10 ng/ml) for 9 days to promote differentiation into macrophages, in the presence or absence of a range of concentrations of imatinib or GW2580. FIG. 12D demonstrates that both imatinib and GW2580 block M-CSF-induced differentiation of human blood monocytes to macrophages. Macrophage were counted based on morphologic features including cytoplasmic inclusions, multipolar process extension, and heterogenous cytoplasmic vacuoles (% macrophage represents the % of total cells with morphologic features characteristic of macrophage). FIG. 12E presents inhibition curves from which IC₅₀ data were generated for imatinib (0.97 μM) and GW2580 (0.01 μM) for Fms GW2580 inhibited M-CSF-induced differentiation of monocytes to macrophages approximately 100 times more potently than imatinib.

FIGS. 13A-13D are graphs showing that the PDGFR, FGFR and Kit inhibitor CGP53716 prevents CIA. Groups of 15 male DBA/1 mice were induced for CIA with type II collagen (CII, 200 mg/mouse) emulsified in CFA, and boosted at day 21 with CII emulsified in IFA. CGP53716 was delivered by daily i.p. administration starting 1 day prior to the induction of CIA. FIG. 13A shows the mean arthritis score of mice dosed with low- and high-dose CGP53716, assessed using a visual arthritis scoring system in the days following primary immunization. FIG. 13B shows the paw thicknesses, in mm, of mice dosed with low- and high-dose CGP53716 in the days following primary immunization. FIG. 13C (scores) and FIG. 13D (paw measurements) show results from a study directly comparing the CIA efficacy of imatinib and CGP53716. Both CGP53716 and imatinib resulted in statistically significant reductions in the severity of CIA. One asterisk indicates a difference of CGP53716 vs. vehicle control of p<0.05, and two asterisks indicate a p<0.01 by Mann Whitney test.

FIGS. 14A-14F show the results of immunohistochemistry (400× magnification) demonstrating differential expression of c-Fms (CSF-1R), PDGFRα and PDGFRβ in human RA synovium. Synovium obtained from a patient with chronic RA at the time of knee replacement was fixed, paraffin embedded, and sections stained with monoclonal or polyclonal antibodies specific for c-Fms (FIG. 14B), PDGFRα(FIG. 14D), or PDGFRβ(FIG. 14F), or the corresponding isotype control antibodies (FIGS. 14A, C, E), respectively, followed by HRP-based detection (Vector Labs). FIG. 14B demonstrates that the anti-c-Fms antibody (Santa Cruz, sc-13949) intensely stained the synovial lining along with less intense staining of the underlying tissue. FIG. 14D demonstrates that the anti-PDGFRα antibody (Santa Cruz, sc-338) stained cells deeper in RA synovial tissue, along with cells near the synovial lining. FIG. 14F demonstrates that the anti-PDGFRβantibody intensely stained a subset of cells underlying the synovial lining (Cell Signaling, #3169).

FIGS. 15A-15E are graphs depicting the flow cytometric identification of mast cell, fibroblast like synoviocyte (FLS), and synovial macrophage populations derived from human RA synovial tissue. Remnant human knee synovium was obtained from an RA patient at the time of arthroplasty, and single cell suspensions generated by enzymatic digestion with Type IV collagenase. The resulting cell suspensions were stained with fluorochrome-conjugated antibodies specific for cell surface markers of hematopoietic cells (CD45) (FIG. 15B), FLS (CD90) (FIG. 15C), mast cells (c-Kit) (FIG. 15D), and synovial macrophages (CD14) (FIG. 15E), along with co-staining with an isotype matched control (FIG. 15A) and anti-MHC class I antibodies. The presented plots represent the MHC class I positive cell populations.

FIGS. 16A-16E are graphs showing that drug combinations between low-dose imatinib and low-dose atorvastatin, rosiglitazone, or enoxaparin may work in synergy to reduce the clinical severity of collagen-induced arthritis. FIG. 16A shows scores from groups of mice that were induced for CIA and treated starting at day 1 with titrations of imatinib (60 mg/kg, 15 mg/kg, 3.75 mg/kg, 0 mg/kg [vehicle-control]) to identify the lower limits of imatinib efficacy, which was a 15 mg/kg dosing regimen. FIG. 16B shows that atorvastatin alone was not effective at preventing CIA at any concentration used (0-20 mg/kg). FIG. 16C shows that 15 mg/kg imatinib and 5 mg/kg atorvastatin, neither of which prevent CIA alone, was highly effective at preventing CIA when used in combination. FIG. 16D shows that low-dose imatinib at 15 mg/kg and low-dose rosiglitazone at 15 mg/kg prevented CIA. FIG. 16E shows that low-dose imatinib at 15 mg/kg and low-dose enoxaparin at 5 mg/kg prevented CIA.

FIGS. 17A-17B are graphs showing that C57BL/6-Kit^{W-sh/W-sh} (Wsh) mice exhibiting defective c-Kit signaling and have significantly reduced mast cells are partially resistant to collagen antibody-induced arthritis. Wsh mice (circles) and control C57BL/6 (C57, diamonds) littermates were induced for collagen antibody-induced arthritis with 1 mg collagen antibodies and were given 50 μg LPS i.p. at day 3 to promote arthritis. FIG. 17A shows that Wsh mice exhibited reduced mean arthritis scores and FIG. 17B shows that Wsh mice exhibited reduced changes in paw thicknesses compared to C57 control mice. These results suggest that Kit and mast cells contribute to the pathogenesis of autoimmune arthritis.

FIGS. 18A-18F are images demonstrates that imatinib treats systemic sclerosis (SSc, scleroderma). In a 24 year old patient with systemic sclerosis, imatinib enabled healing of digital ulcers, resolution of interstitial lung disease, and restoration of skin collagen architecture. A digital ulcer located over the left fourth proximal interphalangeal joint prior to imatinib therapy (FIG. 18A) exhibited significant healing after 3 months of imatinib therapy (FIG. 18B). High resolution computed tomography (HRCT) scan of the chest prior to imatinib therapy demonstrates patchy infiltrates associated with ground glass opacities in the bilateral lower lobes (FIG. 18C), with resolution of ground glass opacities after 3 months of imatinib therapy (FIG. 18D). Hematoxylin and eosin stained skin biopsy of the right arm prior to imatinib therapy showed dense, eosinophilic, tightly packed collagen bundles of the papillary and reticular dermis with an average dermal thickness of 2.81 mm (Magnification 100×) (FIG. 18E), while repeat skin biopsy after 3 months of imatinib taken within 1 cm of initial biopsy shows normalization of collagen architecture, with loose spacing and thinning of collagen bundles and an average dermal thickness of 2.31 mm (FIG. 18F).

FIG. 19 shows structures of the small molecule tyrosine kinase inhibitors SU9518, CGP53716, PD166326 and GW2580. CGP53716 inhibits PDGFR, FGFR and Kit, and provided benefit in the collagen-induced arthritis model for RA (see, e.g., FIG. 13). GW2580 is a highly potent inhibitor of Fms and also inhibits PDGFR (e.g., FIGS. 11B and FIG. 12E), and treated established collagen-induced arthritis (e.g., FIG. 12).

FIG. 20 shows structures of the small molecule tyrosine kinase inhibitors that are FDA-approved for the treatment of various malignancies. These tyrosine kinase inhibitors include imatinib, gefitinib, erlotinib, sorafenib, sunitinib, dasatinib and lapatinib.

### DETAILED DESCRIPTION

### I. Definitions

“Treat” or “treating” means any treatment, including, but not limited to, alleviating symptoms of a disease, disorder, or condition, eliminating the causation of a disease, disorder, or condition on either on a temporary or permanent basis; or slowing, reducing, or inhibiting an ongoing pathological process in an asymptomatic individual. In such an asymptomatic individual, the pathological process would likely eventually cause symptoms. Examples of pathologic processes include but are not limited to autoimmune, inflammatory, or degenerative processes, conditions, or disorders.

“Preventing” refers to inhibiting the initial onset of a pathologic process, such that the pathologic process that could eventually lead to development of symptoms never develops (i.e., preventing the development of a disease, disorder, or condition in a prophylactic manner).

“Therapeutically effective amount” means an amount of a compound that is effective in treating or preventing a particular disorder or condition.

“Pharmaceutically acceptable carrier” is a non-toxic solvent, dispersant, excipient, or other material used in formation of a pharmaceutical composition, i.e., a dosage form capable of administration to a subject or patient.

“Tyrosine kinases” may be abbreviated at “TK,” or similar.

“Tyrosine receptor kinases” may be called “receptor kinases” or abbreviated “RTK,” “TRK,” or similar.

Receptor names may be abbreviated as in the art, for example, “c-Fms” and “c-Kit” may be called “Fms” and “Kit,” and the like.

As used herein, “autoimmune diseases” are a subset of “inflammatory diseases” in which at least a portion of the inflammatory response is directed to autoantigens. Autoimmune diseases are inherently inflammatory disease but the converse relationship is not necessarily true. Numerous examples of these diseases are provided in the text, Figures, and Tables. The present compositions and methods are useful for treating and/or preventing inflammatory diseases, some of which are autoimmune diseases.

As used herein, “single compound” refers to an active compound, and includes the respective pro-drug (if any), active drug, and active metabolites (if any).

### II. Treatment Method

A. Overview

In various aspects, methods for treating and preventing inflammatory diseases are described. Such methods include those for inhibiting T lymphocyte and/or B lymphocyte function, inhibiting fibroblast proliferation, inhibiting inflammatory diseases related to mast cells, inhibiting inflammatory diseases involving activated macrophage, and inhibiting inflammatory diseases involving osteoclasts. The methods include administering an inhibitor of a tyrosine kinase at a dosage sufficient to inhibit a target kinase receptor (or receptors), thereby modulating the downstream signaling effects of the kinase receptors, causing a beneficial therapeutic affect on a subject/patient. In some embodiments, the methods and compositions are for inhibiting at least two receptors.

In one embodiment, the tyrosine kinase inhibitor is imatinib. Imatinib is a small-molecule, protein tyrosine kinase inhibitor known to target the gene product of the Philadelphia chromosome Bcr/Abl translocation found in human subjects with chronic myelogenous leukemia (CML). Imatinib is approved for the treatment of Bcr/Abl positive CML and for treatment of c-Kit-expressing gastrointestinal stromal tumors (GIST) (Druker, B. J., et al., N Engl J Med 344:1031-1037, (2001); Demetri, G. D., et al., N Engl J Med 347:472-480, (2002)). Along with inhibiting Abl and Abl-related kinases at submicromolar concentrations, imatinib specifically and potently inhibits a spectrum of other tyrosine kinases including c-Fms (IC₅₀=1.4 μM), c-Kit (IC₅₀=0.1 μM), and PDGFRα/β (IC₅₀=0.1 μM) (Buchdunger, E., et al., Biochim Biophys Acta 1551:M11-18, (2001); Dewar, A. L., et al., Blood 105:3127-3132, (2005); Fabian, M. A., et al., Nat Biotechnol 23:329-336, (2005)).

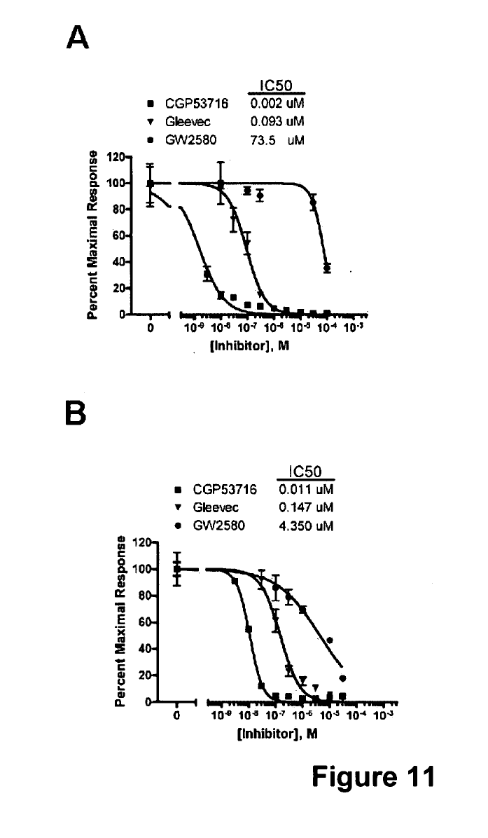

In other embodiments the tyrosine kinase inhibitor has potency for PDGFR and/or c-Kit. For example, CGP53716 is a tyrosine kinase inhibitor that inhibits c-Kit (IC₅₀=0.002 μM, FIG. 11A), PDGFR (IC₅₀=0.011 μM, FIG. 11B), FGFR (IC₅₀=1.1 μM), and Abl (IC₅₀=0.6 μM), but does not inhibit other kinases at clinically relevant concentrations, including EGFR, FGFR, insulin receptors, Src, Lyn, PKA and PKC (Buchdunger, E. et al., Proc Natl Acad Sci USA 92:2558-2562, (1995); Kallio, E., et al., Am J Resp Crit Care Med 160:1324-1332, (1999)). I.P. administration of 100 mg/kg CGP53716 led to plasma levels of 1.6 and 1.9 μM 8 and 24 hours after dosing, respectively (Myllarniemi, M., et al., FASEB J 11:1119-1126, (1997)).

PD166326 is a small molecule tyrosine kinase inhibitor with potency for c-Kit (IC₅₀=0.012 μM) and Abl (IC50=0.002 μM) (Wolff, N., et al., Blood 105:3995-4003, (2005)). PD166326 dosing in mice at 25 and 50 mg/kg led to peak plasma levels of 0.026 and 0.098 μM, and trough levels (15 hours after dosing) of approximately 0.005 and 0.018 μM (Wolff, N., et al., Blood 105:3995-4003, (2005)).

SU9518 is a small molecule tyrosine kinase inhibitor with potency for PDGFR (IC₅₀=0.053 μM) and FGFR (IC₅₀ 4.4) (Yamasaki, Y., et al., Circ Res 88:630-636, (2001); Abdollahi, A., et al., J Exp Med 201:925-935, (2005)). Following an oral dose of 50 mg/kg SU9518 in rats, plasma levels peaked at 1.76 μM and levels were still above 1 μM 8 hours after dosing (Yamasaki, Y., et al., Circ Res 88:630-636, (2001).

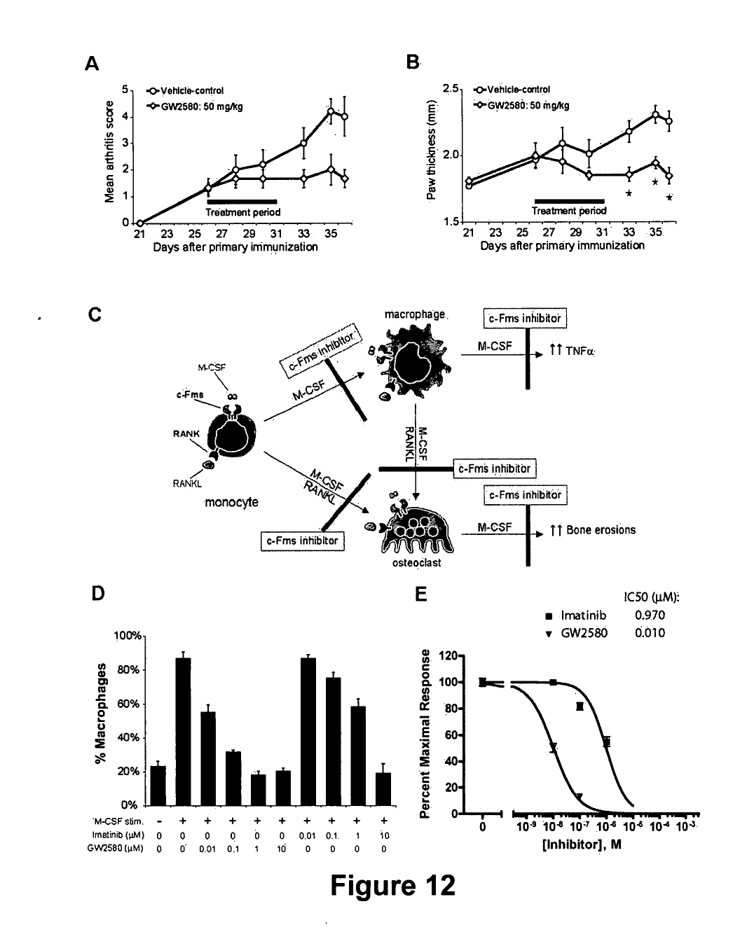

In other embodiments, the tyrosine kinase inhibitor has potency for inhibiting Fms and/or PDGFR. For example, GW2580 is a small molecule that inhibits Fms (IC₅₀=0.01 μM, FIG. 12E) (Conway, J., et al., Proc Nat Acad Sci USA 102:16078-16083, (2005)) and PDGFR (IC₅₀=4.3 μM) (FIG. 11B). GW2580 administered to mice at a dose of 80 mg/kg led to peak plasma concentrations of 5.6 μM (Conway, J., et al., Proc Nat Acad Sci USA 102:16078-16083, (2005)).

The inhibitory profiles for these and other small molecule tyrosine kinase inhibitors are actively being characterized and defined. To date, for the vast majority of kinases (Table 1) and small molecule inhibitors (including the FDA-approved inhibitors presented in Table 2) rigorous in vitro kinase substrate phosphorylation assays (for example, as presented in FIG. 11A) and in vitro cellular response assays (for example, as presented in FIG. 11B) have not been performed. As a result, the inhibitory and IC₅₀ data presented represent the current knowledge in the field, and it is anticipated that further research will identify multiple new and currently undescribed tyrosine kinase inhibitory activities for the small molecule inhibitors described in this application. Further, these new inhibitory specificities may contribute to the efficacy observed in autoimmune and other inflammatory diseases.

B. Imatinib for Treating Rheumatoid Arthritis

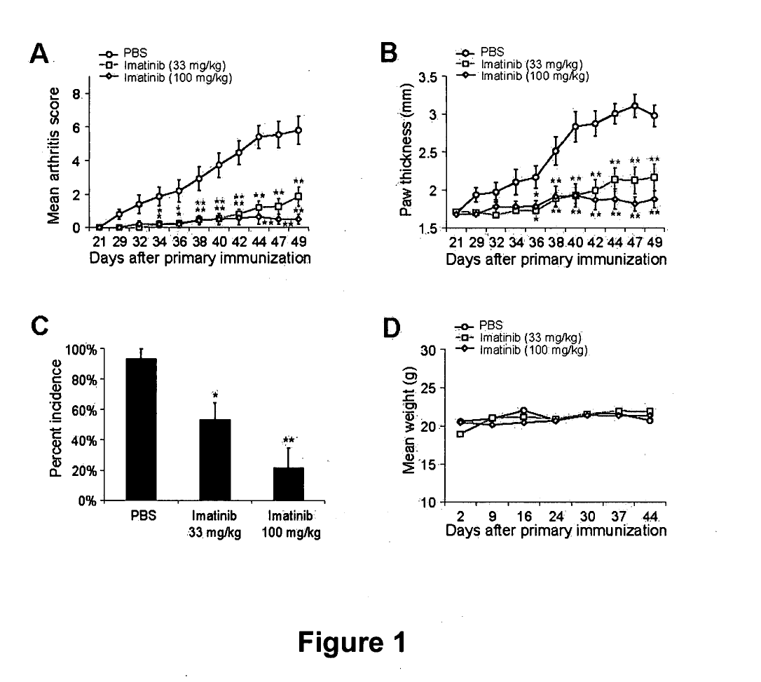

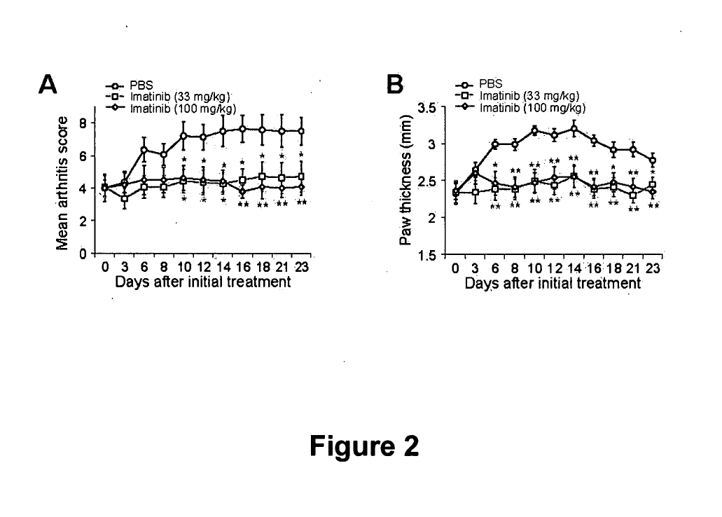

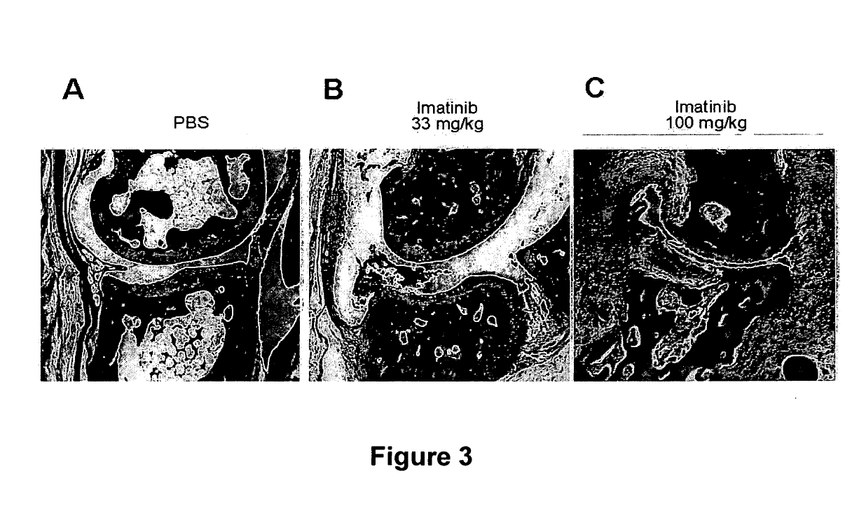

In studies described herein, it is shown that imatinib prevents and treats inflammatory diseases by selectively inhibiting a spectrum of signal transduction pathways central to the pathogenesis of the inflammatory disease. Using collagen-induced arthritis (CIA) in mice as a model of an exemplary inflammatory disease, rheumatoid arthritis (RA), oral administration of imatinib to mice was shown effective to prevent the onset and progression of CIA (e.g., Example 1 and FIGS. 1A-1D). Imatinib was also effective in treating the inflammatory disease CIA in mice with established clinical arthritis (e.g., Example 2 and FIGS. 2A-2B). Histopathologic analysis on the hind paws of the CIA mice treated with imatinib show that the inhibitor reduced synovitis, pannus, and erosion scores in preventing CIA and treating CIA (FIGS. 3A-31).

In vivo and in vitro data, indicate that imatinib potently inhibits diverse cellular responses that play roles in driving synovitis, pannus formation and joint destruction in rheumatoid arthritis. For example, imatinib abrogated PDGFR signaling in human RA patient fibroblast-like synoviocytes, TGF-β mediated Abl signaling in fibroblast cells, c-Kit activation and production of pro-inflammatory cytokines by mast cells, LPS-induced TNFα production by synovial fluid macrophage, and T and B lymphocyte function.

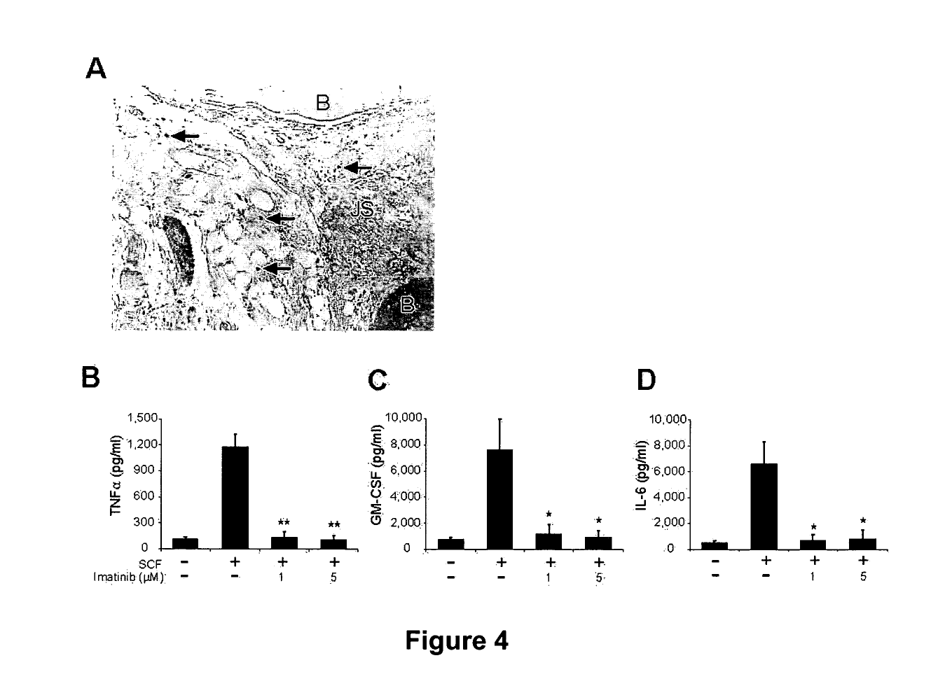

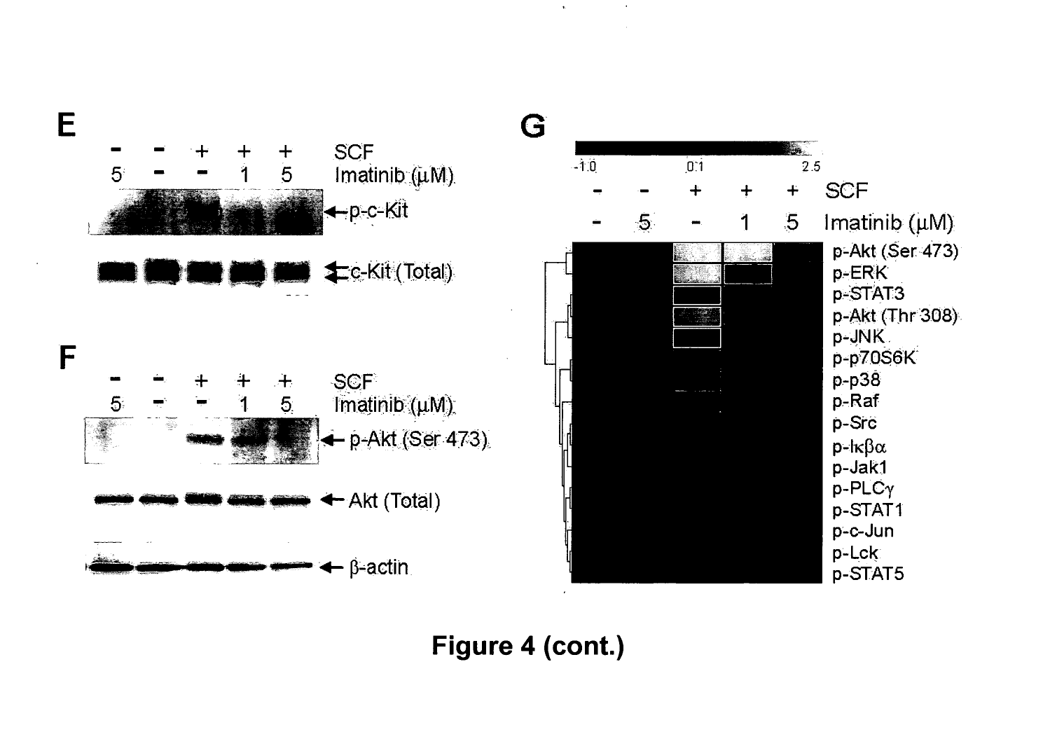

As detailed in Example 3, following confirmation of the presence of mast cells in synovial tissue (FIG. 4A), the mast cells were stimulated with stem cell factor (SCF) in the presence absence or presence of imatinib and cytokine analysis was performed on culture supernatants. As seen in FIGS. 4B-4D, the presence of imatinib reduced mast cell production of TNFα, GM-CSF, and IL-6.

Immunoblotting and reverse phase protein (RPP) lysate array analysis were performed on a mast cell line to characterize tyrosine kinase activation states and signal transduction pathways modulated by imatinib. Immunoblotting results showed that imatinib inhibited stem cell factor-induced phosphorylation of c-Kit (FIG. 4E), with a corresponding reduction in phosphorylation of downstream Akt (FIG. 4F). As seen in FIG. 4G, imatinib prevented phosphorylation of the signaling molecules Akt (e.g., at Ser 473 and Thr 308), P70S6K, and Raf.

The immunoblotting and RPP array technology experiments demonstrated that imatinib inhibits SCF-induced c-Kit phosphorylation and downstream activation of MAPK pathways in mast cells. Further, imatinib inhibited SCF-induced mast cell production of the inflammatory cytokines TNFα, IL-6, and GM-CSF. These data suggest that imatinib-mediated inhibition of mast cell activation could contribute to its efficacy in CIA and potentially human RA.

Taken together, imatinib provides efficacy in CIA by simultaneously inhibiting multiple tyrosine kinases and cellular responses that contribute to the pathogenesis of RA, likely including: (i) PDGFR and c-Abl mediated proliferation of synovial fibroblasts, (ii) c-Fms mediated monocyte maturation into TNFα-producing macrophage and bone degrading osteoclasts, (iii) c-Kit mediated release of TNFα by mast cells, and (iv) c-Abl and Lck mediated activation of B and T cells.

C. Imatinib for Treating Diseases Associated with c-Fms

c-Fms is expressed predominantly on cells of the monocyte lineage and stimulates monocyte differentiation to macrophage and osteoclasts. Fms also regulates macrophage proliferation, differentiation, and survival (Dewar, 2005; Pixley, F. J., and Stanley, E. R., Trends Cell Biol 14:628-638, (2004)).

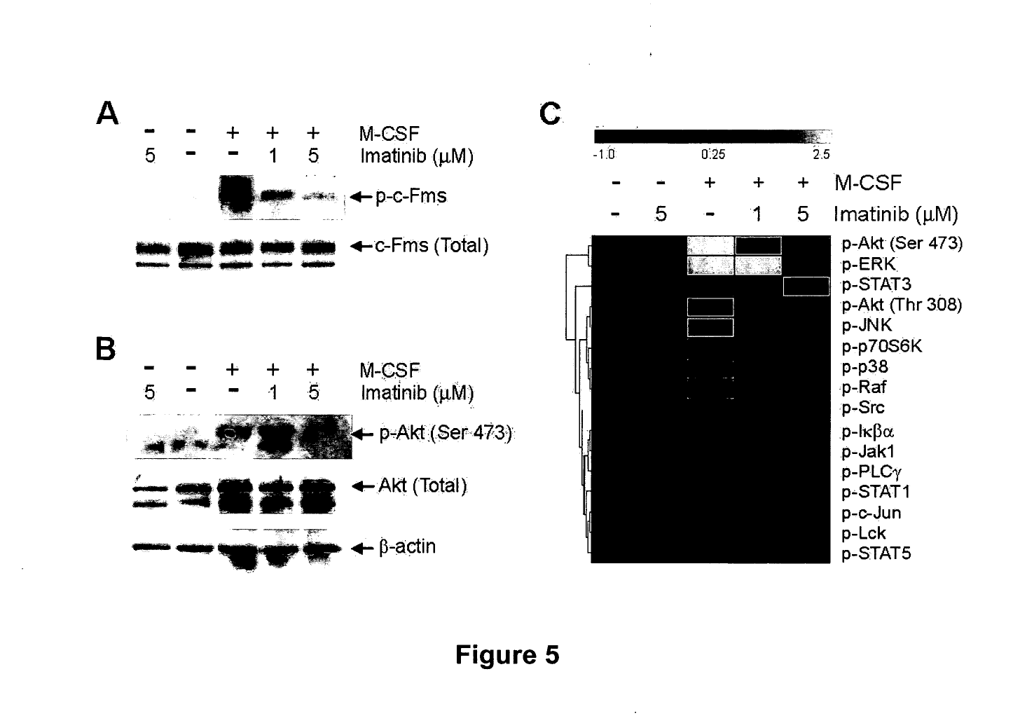

In experiments performed in support of the present compositions and methods, resident peritoneal macrophage cells were isolated from mice, pre-treated with imatinib, and stimulated with M-CSF, as described in Example 5. Lysates were produced for analysis by immunoblotting and RPP array. The immunoblots in FIGS. 5A and 5B show that imatinib inhibited M-CSF-induced phosphorylation of c-Fms, while levels of total c-Fms were similar in all samples (FIG. 5A). The downstream signaling molecule Akt (Ser 473) also exhibited reduced phosphorylation in macrophages pre-treated with imatinib, as shown in FIG. 5B. The RPP array analysis of M-CSF-stimulated macrophage lysates (FIG. 5C) demonstrated that imatinib blocked phosphorylation of protein tyrosine kinases in the MAPK family and other pathways downstream of c-Fms, including Akt (Ser 473 and Thr 308), ERK, STAT3, JNK, P70S6K and p38. The immunoblot and RPP array data demonstrate that imatinib potently inhibits M-CSF-induced macrophage activation through c-Fms.

These results suggest that inhibiting the differentiation of monocytes into macrophage, a process known to produce TNFα, can be used to modulate, treat, or prevent inflammatory disease, such as rheumatoid arthritis, psoriasis, psoriatic arthritis, Crohn's disease, ankylosing spondylitis, and systemic lupus erythematosus (SLE). Imatinib-mediated inhibition of differentiation of monocytes into osteoclasts, could also prevent osteoclast-mediated bony destruction in RA, psoriatic arthritis, ankylosing spondylitis, and other inflammatory arthritidies.

D. Imatinib for Treating Diseases Involving TKs of B Cells and T Cells

Imatinib also inhibits c-Abl expressed in B cells and Lck expressed in T cells, and can thereby attenuate adaptive autoimmune responses in disease, such as rheumatoid arthritis. The presence of anti-citrulline antibodies in rheumatoid arthritis, the contribution of anti-citrulline antibodies to destructive synovitis in rodent models of arthritis, and the efficacy of anti-CD20 therapy, all suggest an important role for B cells in the pathogenesis of rheumatoid arthritis (Firestein, G. S., Nature 423:356-361, (2003); Kuhn, K. A., et al., J. Clinical Investigation).

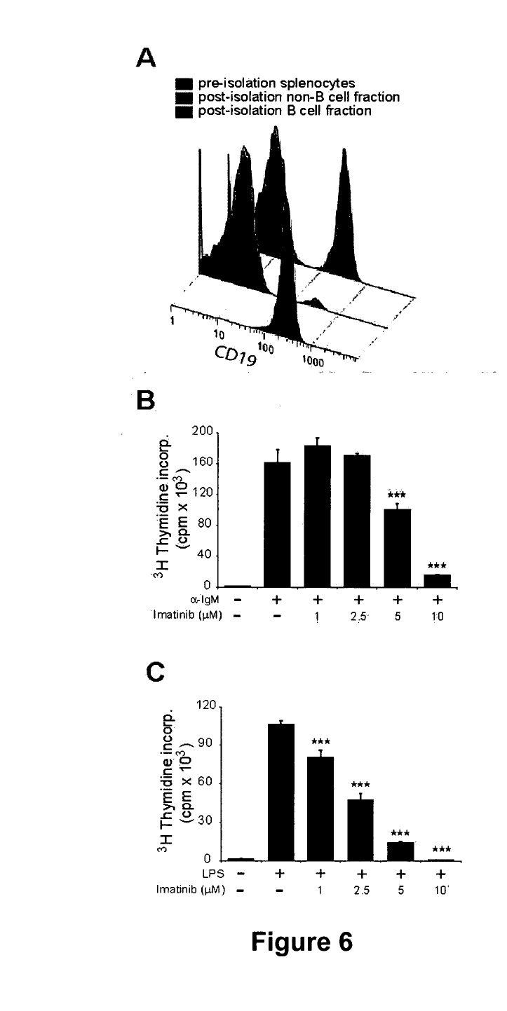

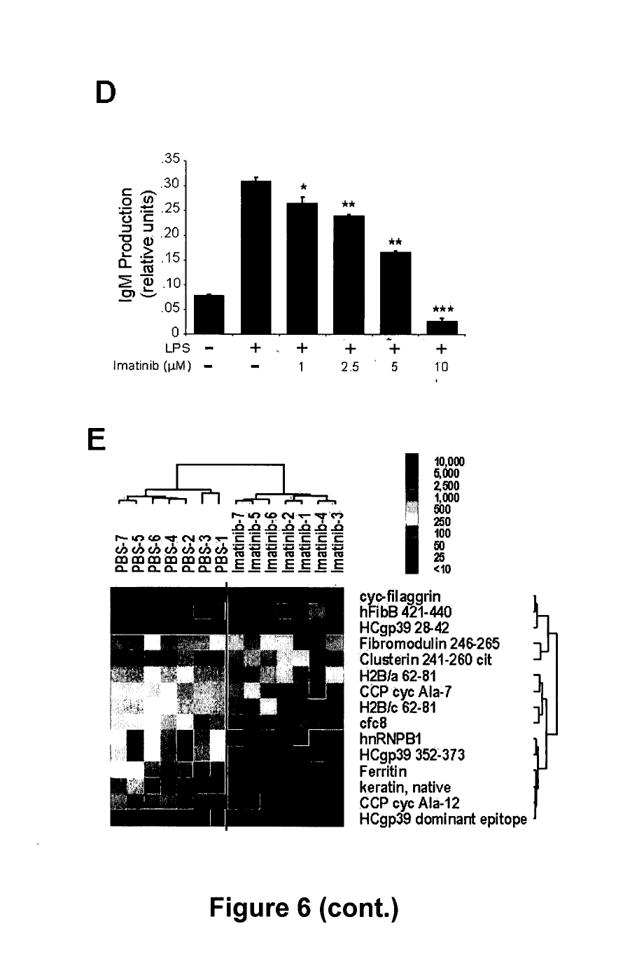

In the present studies, epitope spreading of anti-synovial B cell responses was reduced in CIA mice treated with imatinib (Example 5 and FIGS. 6A-6E). This difference may have been due to the effect of imatinib on c-Abl. c-Abl phosphorylates and colocalizes with CD19 on the B cell surface following stimulation of the B cell antigen receptor (BCR), and c-Abl deficient mice have defective BCR signaling (Zipfel, P. A., et al., J Immunol 165:6872-6879, (2000)). Since imatinib blocks c-Abl kinase activity at sub-micromolar concentrations (Buchdunger, 2001; Dewar, 2005; Fabian, 2005), it is possible that imatinib reduces expansion of autoreactive B cell responses in CIA by inhibiting BCR signaling.

A role for autoreactive T cells in RA is supported by the presence of T cell infiltrates in rheumatoid synovium, the association of the shared epitope HLA-DR4 polymorphism with RA, and the efficacy of CTLA4-1 g (Firestein, 2003; Genovese, M. C., et al., N Engl J Med 353:1114-1123, (2005)). In vitro studies suggest that imatinib attenuates T-cell activation via inhibition of the TCR-associated tyrosine kinase Lck (Dietz, A. B., et al., Blood 104:1094-1099, (2004)). The in vitro studies described herein (Example 6) demonstrate that imatinib inhibited anti-CII T cell proliferation at 10 μM, the highest concentration tested.

E. GW2580 for Treating Diseases Involving Fms and PDGFR

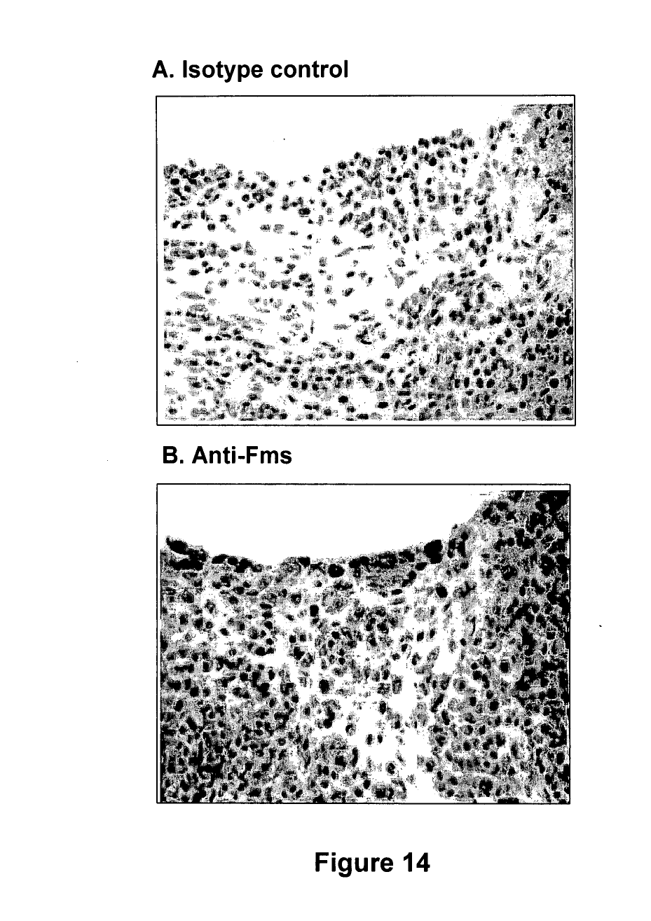

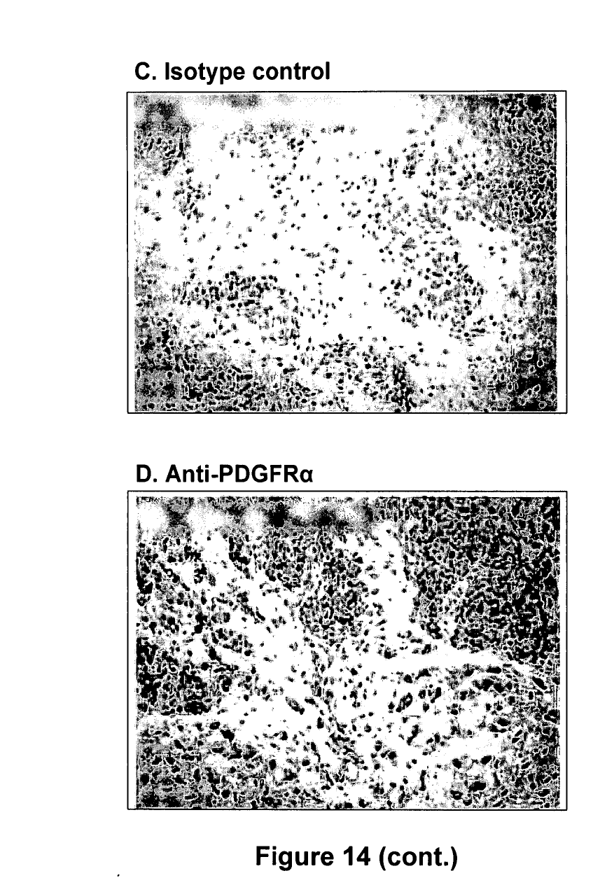

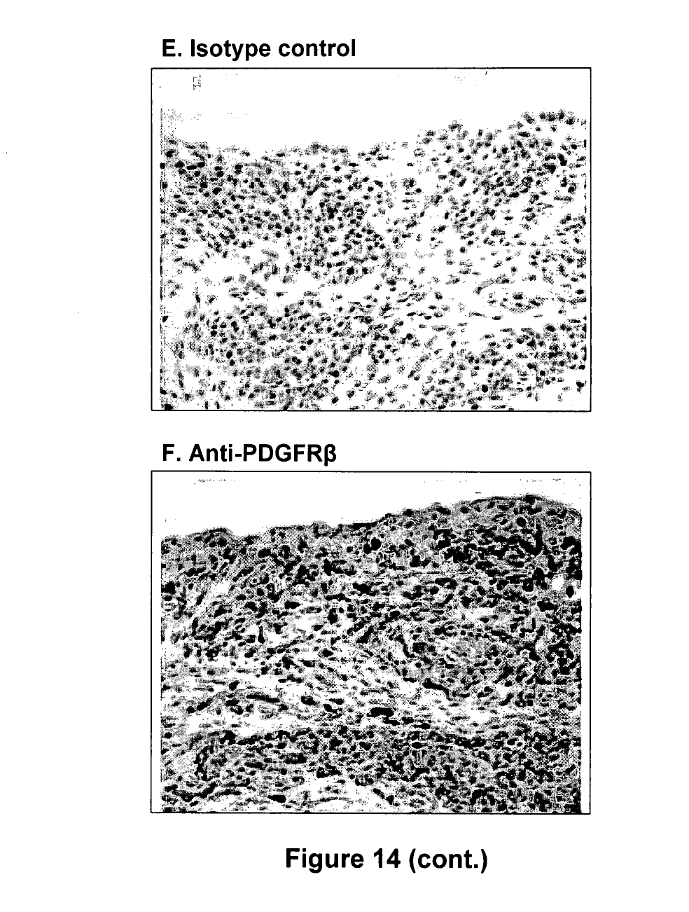

In another embodiment, a small-molecule tyrosine kinase inhibitor that is highly potent for Fms and also inhibits PDGFR provides efficacy in treating the collagen-induced arthritis (CIA) model for RA. The data shown in FIG. 12 and described in Example 9 demonstrating that an Fms and PDGFR inhibitor, GW2580, treats established collagen-induced arthritis. Further, it is demonstrated that this inhibitor blocks differentiation of monocytes into macrophage capable of producing TNFα and other pro-inflammatory cytokines. Immunohistochemistry experiments demonstrate that Fms and PDGFR expressed at high levels in the synovial lining, as well as at a moderate level deeper in synovium derived from RA patients (FIG. 14 and Example 11). GW2580 likely also provides benefit by inhibiting Fms-mediated differentiation and activation of osteoclasts, which metabolize and destroy bone in CIA and RA. GW2580's inhibition of PDGFR likely also contributes to its efficacy in CIA, by inhibiting the proliferation of synovial fibroblast to form invasive pannus tissue.

F. CGP53716 for Treating Diseases Involving PDGFR, FGFR and Kit

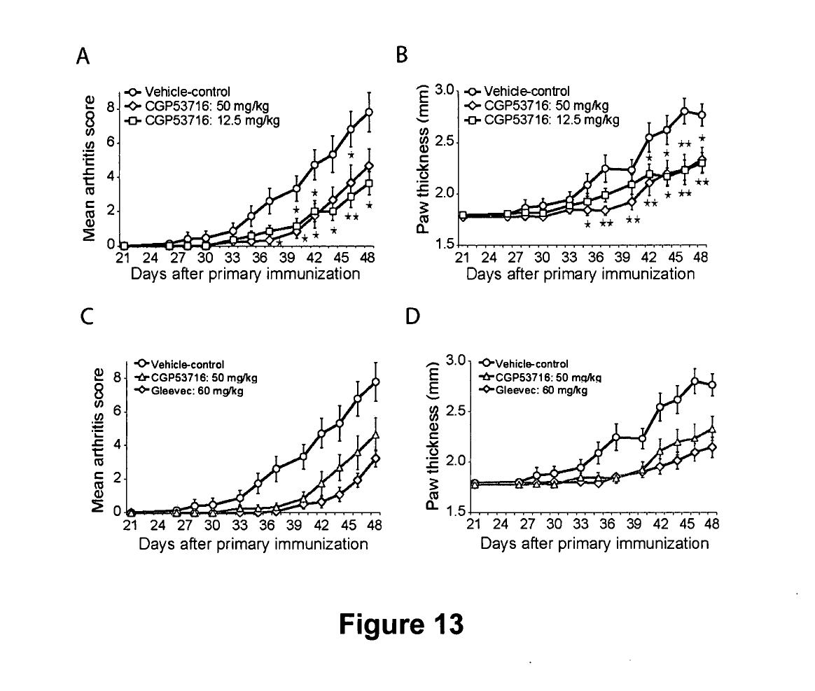

In yet another embodiment, a small-molecule tyrosine kinase inhibitor that inhibits PDGFR, FGFR and Kit provides efficacy in preventing the induction of RA. As shown in FIG. 13 and described in Example 10, the PDGFR, FGFR and Kit inhibitor, CGP53716, prevents mice from developing CIA. PDGFR and FGFR mediate proliferation of synovial fibroblasts, and CGP53716 likely provides benefit by preventing hyperplasia of the synovial lining fibroblasts to form pannus tissue that invades adjacent cartilage and soft tissues. Additionally, by inhibiting Kit, CGP53716 blocks mast cell activation and inflammatory mediator release. Immunohistochemistry demonstrated that both PDGFRα and PDGFRβ are expressed in synovium derived from patients with RA (FIG. 14 and Example 11).

G. Imatinib for Treating Multiple Sclerosis

In further experiments performed in support of the present compositions and methods, the ability of imatinib to treat and/or prevent another exemplary inflammatory condition, experimental autoimmune encephalomyelitis (EAE), was evaluated. EAE is a widely used animal model for multiple sclerosis (MS).

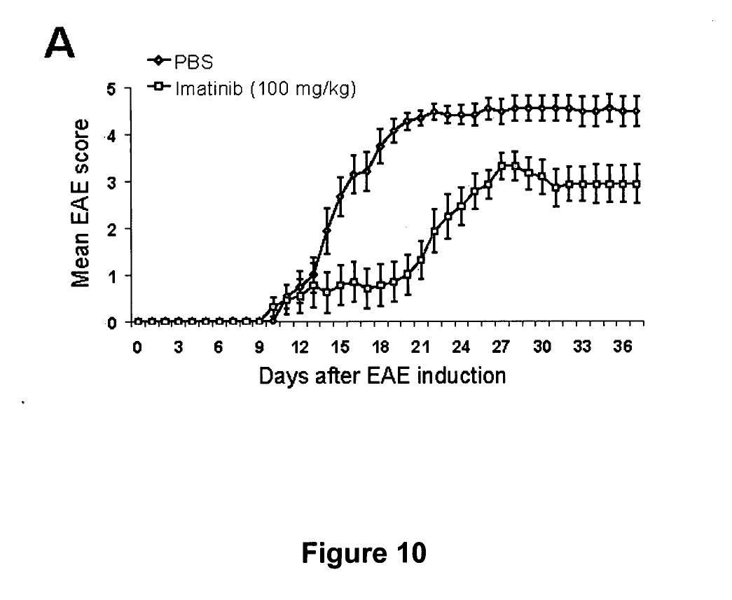

EAE mice were treated with imatinib twice daily and the severity of the disease was determined using a standard scoring system, described in Example 8. The results are shown in FIG. 10. Animals treated with imatinib demonstrated delayed onset and reduced severity of EAE compared to control mice. Imatinib likely provided a beneficial therapeutic effect by (i) inhibiting Fms-mediated monocyte differentiation into macrophage, and priming of macrophage to produce TNFα; (ii) inhibiting Kit-mediated mast cell inflammatory mediator release; (iii) inhibiting T and B cell function via Lck and Abl, respectively; (iv) inhibiting gliosis that is possibly mediated by PDGFR and Abl; and/or (v) a combination of these effects. Simultaneous inhibition of multiple tyrosine kinases is likely important for the mechanism of action of the present compositions and methods.

H. Imatinib for Treating Systemic Sclerosis

The pathogenesis of systemic sclerosis (SSc, scleroderma) involves fibrosis, vasculopathy, inflammation, and autoimmunity. Activation of profibrotic pathways in SSc involves over-expression of the cytokines transforming growth factor (TGF)-beta (TGF-β) and platelet derived growth factor (PDGF). PDGF receptors are upregulated in the skin and bronchoalveolar lavage fluid of patients with SSc and, when activated, lead to fibroblast and myofibroblast proliferation (Yamakage, A. et al. (1992) J. Exp. Med. 175:1227-1234; Ludwicka, A. et al. (1995) J. Rheumatology 22:1876-83). In addition, PDGFR may be implicated in the initiation of the inflammatory response in SSc, through stimulating the production of monocyte chemoattractant protein 1 (MCP-1) (Distler, O. et al., (2001), Arthritis Rheum. 44:2665-78). A recent report showed that SSc patients have autoantibodies against PDGFR, which stimulate the production of reactive oxygen species and type I collagen expression, consistent with the vasculopathic and fibrotic features of the disease (Baroni, S. (2006) N. Engl. J. Med. 354:2667-76).

TGF-β signaling is an important profibrotic pathway in systemic sclerosis (Distler, J., et al., Arthritis Rheum 56:311-333 (2007)). TGF-β stimulation in fibroblasts signals through c-Abl (Wang, S., et al., FASEB J 19:1-11 (2005)), and signaling through Abl likely contributes significantly to the fibrosis of the skin, lungs, kidneys and other organs in systemic sclerosis.

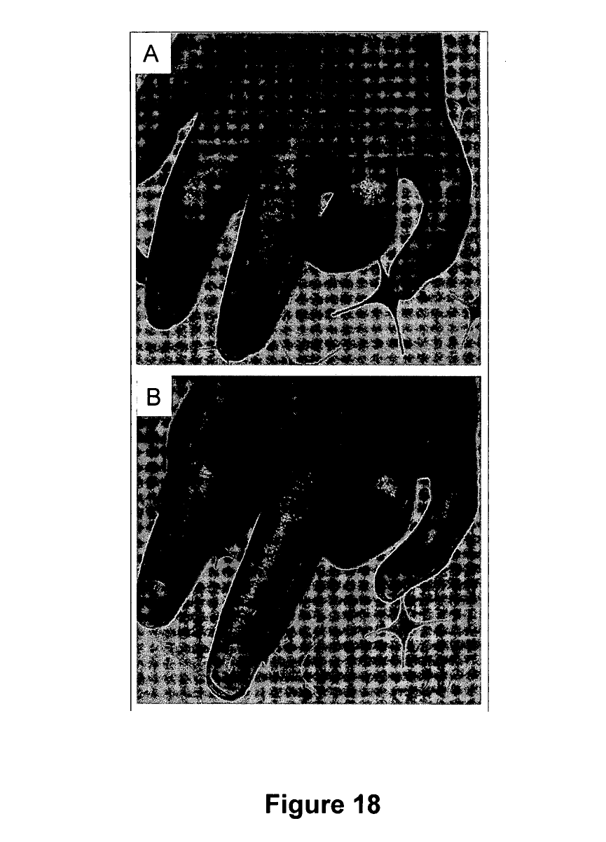

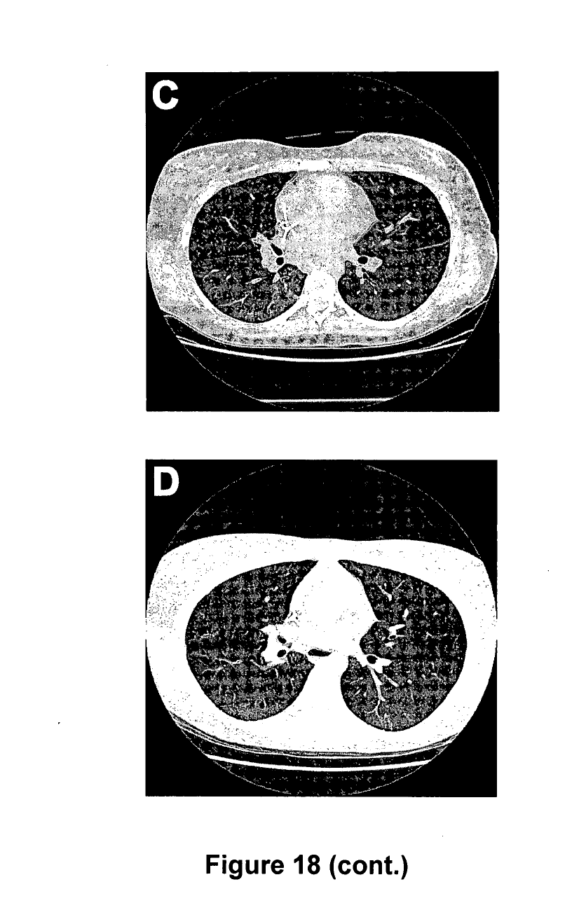

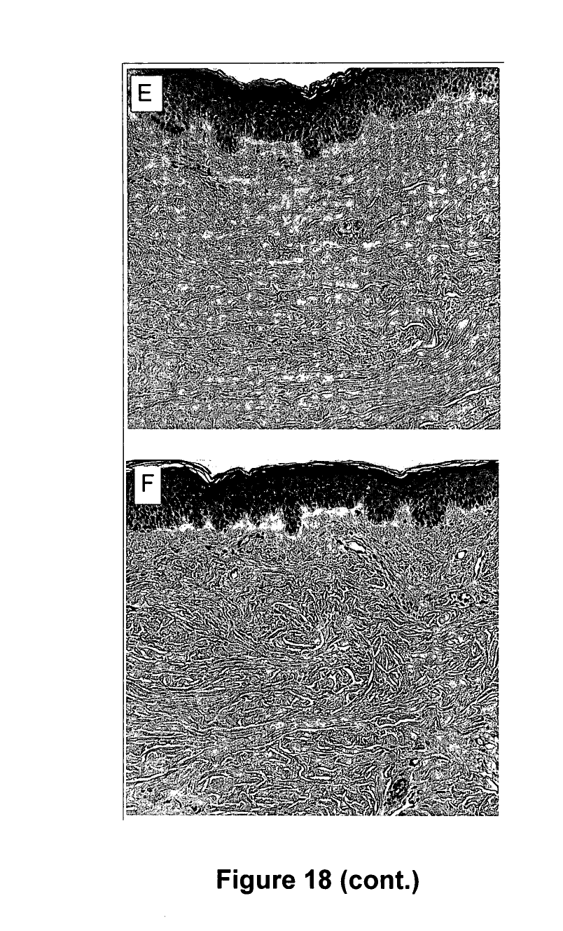

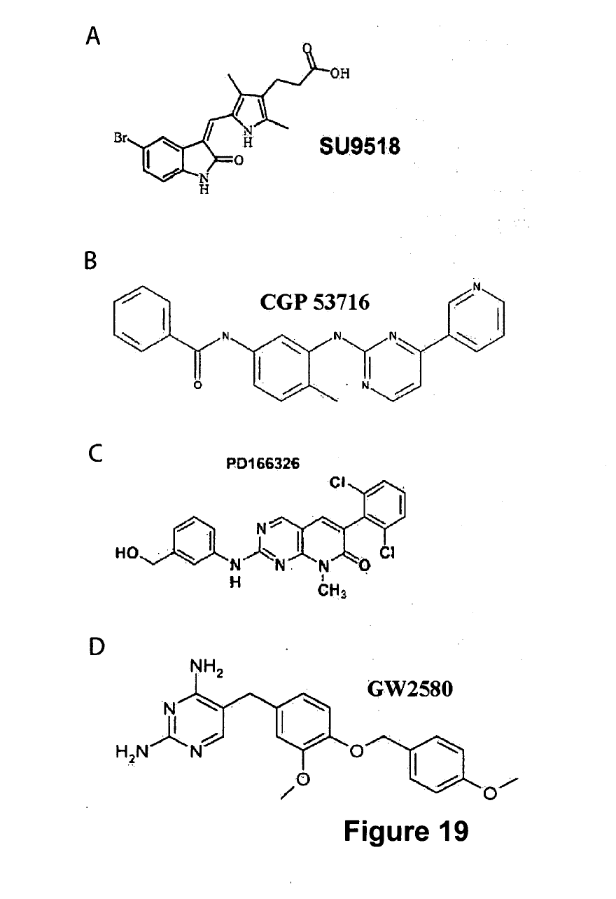

A patient with early diffuse SSc experienced improvement in cutaneous, pulmonary, and musculoskeletal manifestations of her disease in response to treatment with imatinib mesylate (Gleevec™, Novartis, East Hanover, N.J.) therapy (Example 15 and FIG. 18A, C, E (before therapy) and 18B, D, F (after therapy)). Imatinib likely provided a beneficial therapeutic effect by (i) inhibiting PDGFR- and/or Abl-mediated proliferation of skin, lung and other tissues; (ii) inhibiting Fms-mediated monocyte differentiation into macrophage, and priming of macrophage to produce TNFα; (iii) inhibiting Kit-mediated mast cell inflammatory mediator release; (iv) inhibiting T and B cell function via Lck and Abl, respectively; and/or (v) a combination of these effects. Simultaneous inhibition of multiple tyrosine kinases and the pathogenic cellular responses they mediate is likely important for the efficacy of imatinib in treating systemic sclerosis.

G. Conclusion

Based on the foregoing, it can be seen that administration of a tyrosine kinase inhibitor, exemplified by imatinib, GW2580, SU9518, PD166326, and CGP53716, is contemplated for prevention and treatment of patients with a number of inflammatory diseases.

In one embodiment, the disease is severe RA, particularly RA refractory to treatment with methotrexate, TNF-antagonists, or other disease-modifying anti-rheumatic drugs. In the studies described herein, imatinib effectively treated CIA and inhibited multiple signal transduction pathways that drive pathogenic cellular responses in rheumatoid arthritis. In other embodiments, the inflammatory disease is an autoimmune diseases, including but not limited to not to psoriasis, psoriatic arthritis, nephritis, glomerulonephritis, multiple sclerosis, inflammatory bowel disease, systemic lupus erythematosus, autoimmune diabetes, scleroderma, Crohn's disease, etc.

While imatinib-mediated inhibition of macrophage maturation and TNFα production, mast cell activation and TNFα release, and autoreactive B and T lymphocyte activation could provide therapeutic benefits in many autoimmune diseases, the ability of imatinib to inhibit PDGFR and Abl make it particularly suited for the treatment of rheumatoid arthritis, scleroderma and other diseases in which fibrotic processes play a role in pathogenesis.

Without being limited to a theory, the present compositions and methods appear to produce a beneficial effect by inhibiting particular TKs involved in the pathogenesis of an inflammatory disease. Such TKs may be directly involved in the inflammatory responses and/or involved in host cell responses to inflammation.

Examples of tyrosine kinases directly involved in autoimmune and inflammatory responses include: (i) c-Fms-mediated monocyte maturation and macrophage priming to produce TNFα; (ii) c-Kit-mediated mast cell activation and pro-inflammatory mediator release; and (iii) Abl or Lck-mediated lymphocyte activation. These kinases directly contribute to autoimmune or inflammatory responses that cause organ, tissue, or cell injury or inflammation.

In the case of host cell responses, there are a variety of aberrant host cell responses to inflammation and tissue injury that contribute to the clinical phenotype of autoimmune and other inflammatory diseases. Such host cell responses include PDGFR-mediated, FGFR-mediated, and Abl-mediated fibroblast proliferation, as observed in the formation of pannus in RA or skin tightening in scleroderma. Dysregulated host cell responses that can include fibrotic-like responses are observed in many other autoimmune and inflammatory diseases including: multiple sclerosis in which gliosis occurs in damaged white matter; systemic lupus erythematosus in which glomerulosclerosis occurs in damaged glomeruli; autoimmune hepatitis and primary biliary cirrhosis in which significant liver and biliary fibrosis are observed; idiopathic pulmonary fibrosis which is characterized by lung fibrosis; and Crohn's diseases in which fibrosis of the bowel wall and mesentery occurs.

In addition to highly selective tyrosine kinase inhibitors, it is anticipated that tyrosine kinase inhibitors that inhibit select subsets of kinases involved in the pathogenesis of inflammatory diseases will provide more significant benefit.

Examples of inhibitors that inhibit multiple tyrosine kinases involved in the pathogenesis of autoimmune and inflammatory diseases include imatinib, which inhibits Fms, Kit, PDGFRα, PDGFRβ, and Abl; CGP53716, which inhibits PDGFR, FGFR and c-Kit; and GW2580, which inhibits Fms and PDGFR.

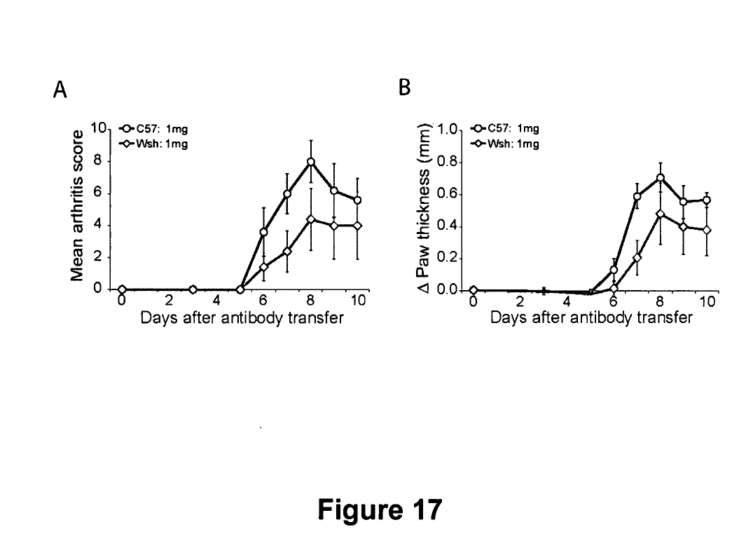

It is likely that inhibition of a single tyrosine kinase may only provide modest and limited efficacy in treating rheumatoid arthritis, psoriasis, Crohn's and other autoimmune diseases. This is supported by the observations that genetic deficiencies in individual tyrosine kinases (or their cognate ligands) against which imatinib and other TK inhibitors act only reduces autoimmune arthritis to a modest degree. For example, C57BL/6Kit^{W-sh/W-sh} (Wsh) mice exhibiting defective c-Kit signaling exhibit only a mild reduction in the severity of arthritis (FIG. 17). Additionally, osteopetrotic (op/op) mice on the C57BL/6×C3HeB/FeJ background are deficient for the Fms-ligand M-CSF and exhibit only a modest resistance to autoimmune arthritis (Campbell, I., et al., J Leuk Biol 68:144-150 (2000)). These data suggest that genetic mutations in c-Kit or the Fms-ligand M-CSF are insufficient to fully protect mice against autoimmune arthritis, and thus that inhibition of individual tyrosine kinases will likely be insufficient to treat autoimmune arthritis and other autoimmune diseases.

The likelihood that inhibition of a single tyrosine kinase will only provide modest and limited efficacy in the treating rheumatoid arthritis, psoriasis, Crohn's and other autoimmune diseases is also supported by observations made in current clinical practice, in which combinations of drugs are frequently needed to adequately treat these autoimmune diseases. In the case of RA, hydroxycholoquine is frequently used in combination with methotrexate. An anti-TNF agent (adalimumab, etanercept or infliximab) is added where patient fail to adequately respond to hydroxycholoquine with methotrexate. Similar combination therapy approaches are used to treat other diseases. For example, in Crohn's disease, sulfasalazine and anti-TNF agents are frequently used in combination. In SLE and other rheumatic diseases, prednisone is used in combination with imuran, cytoxan or mycophenolate mofetil. Based, in part, on these observations relating to conventional therapy, in combination with the results described herein, it is anticipated that compounds that inhibit multiple tyrosine kinases involved in pathogenesis will provide significant clinical efficacy.

Based on shared structural features among ATP-binding sites of tyrosine kinases, many small-molecule tyrosine kinase inhibitors that directly interact with the ATP-binding site are likely to cross-inhibit several tyrosine kinases (Table 1). Consequently, a small-molecule tyrosine kinase inhibitor with the most appropriate inhibitory profile can be selected for the treatment of a disease characterized by aberrant expression or activity of particular tyrosine kinases. Ideally, such a tyrosine kinase inhibitor would be a single active compound that cross-inhibits the relevant and pathogenesis-driving kinases.

The exemplified inhibitors are imatinib (which inhibits c-Fms, c-Kit, PDGFRα, PDGFRβ, and Abl), SU9518 (which inhibits PDGFR and FGFR), GW2580 (which inhibits Fms and PDGFR), CGP53716 (which inhibits PDGFR, FGFR and Kit), and PD166326 (which inhibits Kit and Abl). The structures of these compounds are shown in FIG. 19. Further information regarding these compounds is provided in Table 5.

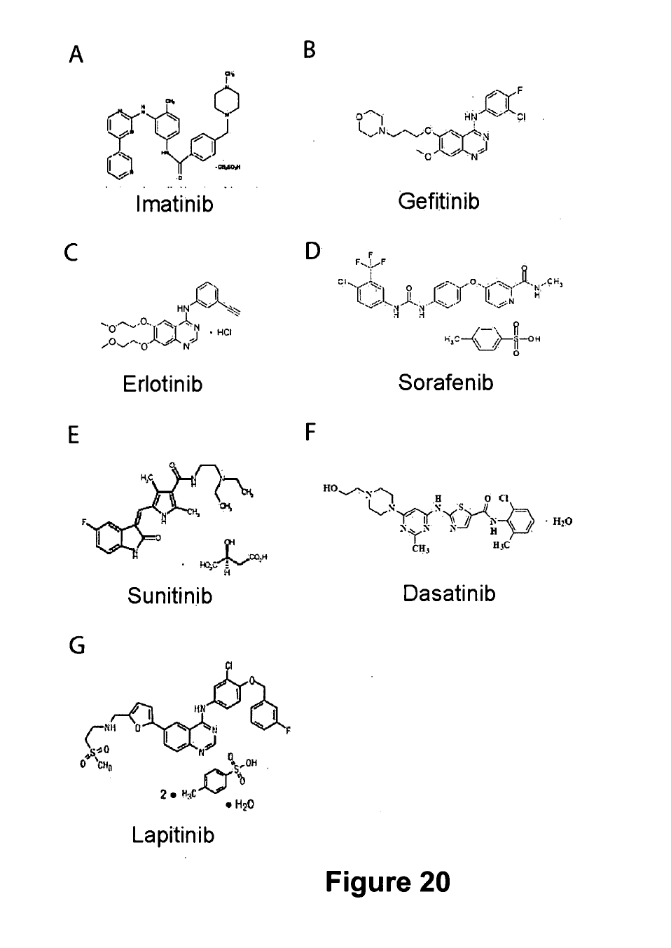

FIG. 20 shows the structures of additional compounds known to inhibit small-molecule tyrosine kinase inhibitors, and which may work in the present compositions and methods. Further information relating to these compounds is provided in Tables 6 and 7).

Additional tyrosine kinase inhibitors likely to provide efficacy in treating or preventing a human autoimmune disease can be identified by, for example:

(i) rational selection of a tyrosine kinase inhibitor compound demonstrated to inhibit one, or preferably at least two, tyrosine kinases involved in the pathogenesis of a particular autoimmune or other inflammatory disease, and

(ii) demonstrating that the tyrosine kinase inhibitor compound can treat established disease in the rodent model relevant to the autoimmune or other inflammatory disease.

The mechanisms known to mediate human inflammatory diseases can help guide and inform selection of tyrosine kinase inhibitors that might provide efficacy in a particular autoimmune or other inflammatory disease, for example:

(i) knowledge of the prominent role for TNFα in rheumatoid arthritis, psoriasis and Crohn's suggests that (a) Fms-inhibitors and other inhibitors that inhibit differentiation of monocytic cells to TNFα-producing macrophage (for example, see FIG. 12), and (b) Kit inhibitors that block mast cell production and release of TNFα and other pro-inflammatory mediators (for example, see FIG. 4), could be effective in treating particular diseases;

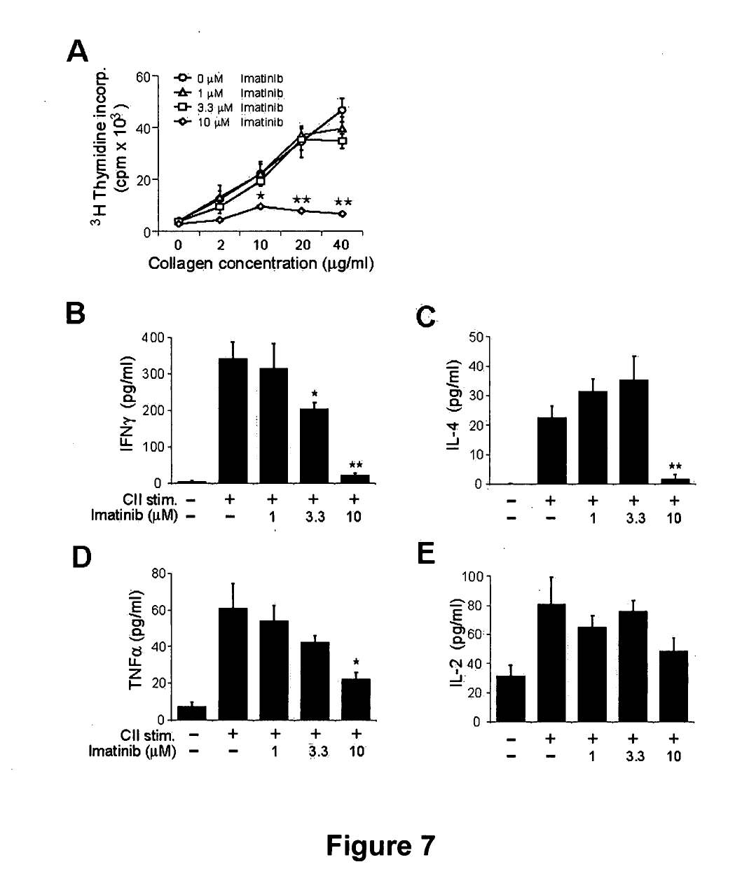

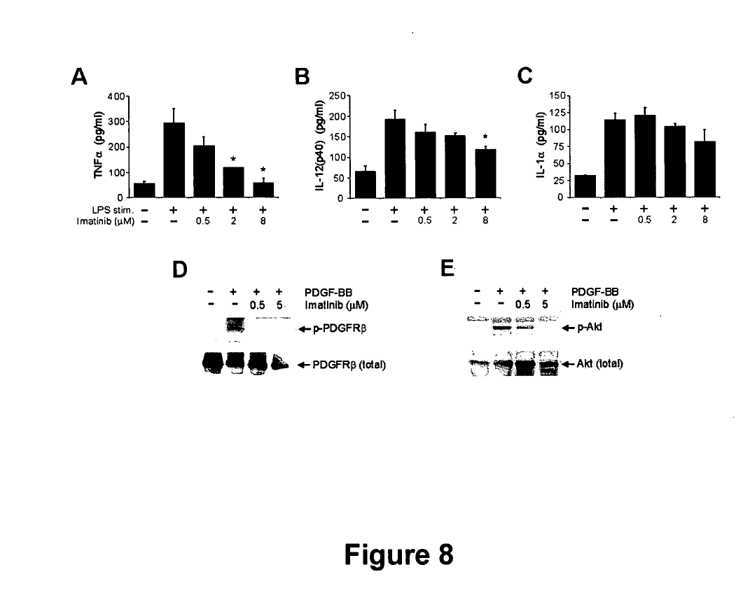

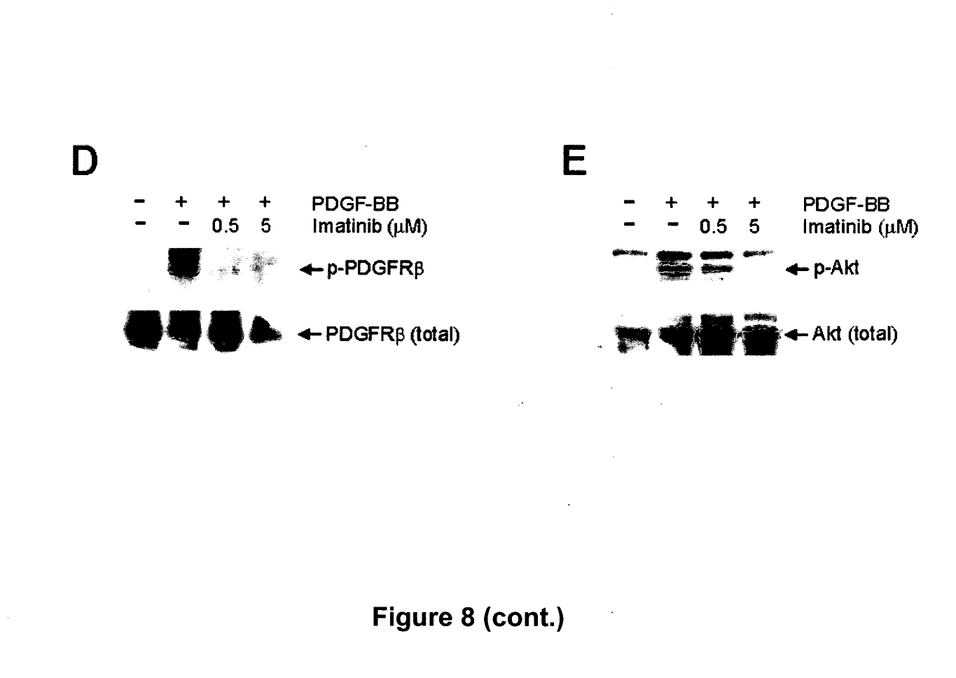

(ii) knowledge of a prominent role for fibroblast proliferation or fibrotic-like host cell responses in the clinical phenotype. Such fibrotic-like responses are observed in RA (proliferation of fibroblast-like synoviocytes to form invasive pannus, see FIG. 8), scleroderma (proliferation of skin fibroblasts to cause tight skin), idiopathic pulmonary fibrosis (fibrosis causing lung dysfunction), Crohn's disease (bowel fibrosis contributes to symptoms), multiple sclerosis (characterized by gliosis and scaring of damaged white matter), and systemic lupus (involving glomerulosclerosis and scaring of the kidney) and suggest that PDGFR, FGFR and Abl inhibitors could be effective in treating particular diseases; and (iii) knowledge of the involvement of autoimmune B and T cells in the inflammatory disease suggest that Abl- and Lck-inhibitors, which mediate inhibition of B and T cells, respectively, could be effective in treating particular diseases (for example, see FIGS. 6 and 7).

Similarly, while the exemplary tyrosine kinase targets are PDGFR, FGFR, Fms, Kit, and Abl, it may be desirable to target other tyrosine kinases. One skilled in the art will appreciate that the active compound may be an active drug, a pre-drug (if any), or an active metabolite (if any) that mediate kinase inhibition as described herein. Alternatively, several TK inhibitors, each specific for tyrosine kinases involved in pathogenesis, could be administered in combination.

### IV. Delivery and Formulations

As compared with the tyrosine kinase inhibitor doses required to treat CML, GIST and other malignancies, lower doses of imatinib and other small molecule tyrosine kinases inhibitors could provide benefit in autoimmune diseases. CML and GIST arise from primary mutations in Abl and Kit, and thus require relatively high doses of imatinib to inhibit proliferation of the malignant cells. By contrast, inflammatory diseases are generally not associated with mutations in these kinases, and wild-type kinases participate in the dysregulated cellular responses that mediate inflammation, tissue injury, and aberrant host cell responses. The IC₅₀ of imatinib and other tyrosine kinase inhibitors for wild-type kinases is lower than that of the mutated kinases associated with malignancy, and it is anticipated that lower dosing regimens may provide benefit in autoimmune and other inflammatory diseases.

The dose of imatinib, GW2580, SU9518, PD166326, CGP53716 or other tyrosine kinase inhibitor is selected by an attending medical caregiver according to means known in the art, including but not limited to the disease to be treated, the severity of the disease, and the condition of the patient. In one embodiment, imatinib is administered at least once per day, more preferably at least twice per day. A dose of 200 mg once per day, more preferably of 400 mg once per day, and even 600 mg once per day, are contemplated. In one embodiment, a dose of imatinib for treatment of certain inflammatory diseases such as RA, is given at a dose lower than the currently approved dose. In particular, a dose of 100 mg/day or less, 50 mg/day or less, or 25 mg/day or less is considered.

Administration less than every day is also contemplated, for example administration every other day or several times per week. Additionally, intermittent courses of therapy with imatinib or another tyrosine kinase inhibitor are contemplated, for example, treatment for one week then off drug for one week, or treatment for one week then off drug for three weeks, or treatment only during periods of disease flare.

In a preferred embodiment, the tyrosine kinase inhibitor is administered orally. Solid dosage forms for oral administration include capsules, tablets, pills, powders, and granules. In such solid dosage forms, the active compound is mixed with at least one inert, pharmaceutically-acceptable excipient or carrier, such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules may be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. The active compound can also be in micro-encapsulated form, if appropriate, with one or more of the above-mentioned excipients.

Liquid dosage forms for oral administration include pharmaceutically-acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents. Suspensions may contain, in addition to the active compounds, suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

From the foregoing, various additional aspects and embodiments of the present compositions and methods will become apparent. The following Examples are provided to illustrate the compositions and methods but are not intended as limiting.

### V. EXAMPLES

The following examples are illustrative in nature and are in no way intended to be limiting.

Methods

A. Cell lines and Antibodies. The mouse mast cell line C1.MC/57.1 was provided by Dr. S. Galli (Young, J. D., et al., Proc Natl Acad Sci USA 84:9175-9179, (1987); Tsai, M., et al., FASEB J. 10:A1253, (1996)). Antibody sources: anti-c-Fms and PDGFRβ (Santa Cruz Biotechnology); anti-β-actin (Sigma), and other antibodies (Cell Signaling Technology).

B. Animals. Six to eight week-old male DBA/1 mice (Jackson Laboratory) were used in protocols approved by the Stanford University Committee of Animal Research and in accordance with the National Institutes of Health (NIH) guidelines. Mice expressing a TCR specific for CII were provided by Dr. W. Ladiges (University of Washington) (Osman, G. E., et al., Int Immunol 10:1613-1622, (1998)). Wsh mice on the C57 background and wild-type C57 mice were provided by Dr. S. Galli (Stanford University).

C. Human RA synovial fluid and tissue samples. Human synovial fluid and tissue samples were collected under Stanford University Institutional Review Board approved protocols and after provision of informed consent from patients with the diagnosis of RA based on the revised criteria of the American College of Rheumatology.

D. Collagen-induced arthritis studies. CIA in DBA/1 mice was induced and scored as described (Coligan, J. E., et al., CURRENT PROTOCOLS IN IMMUNOLOGY, Hoboken, N.J., John Wiley and Sons, Inc. 15.15.11-15.15.24, (1994)). Imatinib tablets (purchased from Stanford Hospital Central Pharmacy) were ground and diluted in PBS, and 33 mg/kg or 100 mg/kg delivered by oral gavage twice daily, starting on the day prior to CIA induction in prevention experiments and following the development of clinical arthritis in treatment experiments. CGP53716 was synthesized and provided by Dr. Darren Veach (Memorial Sloan-Kettering Cancer Center). GW2580 was purchased from Calbiochem (Catalog #344036).

E. Histopathology studies. Hind limbs were fixed and decalcified in CalEx II (Fischer Scientific), embedded in paraffin, and H&E stained sections scored for synovitis, pannus, and bone and/or cartilage destruction (Deng, G. M., et al., Nat Med 11:1066-1072, (2005)).

F. Isolation and stimulation of RA synovial cells. RA synovial fluid mononuclear cells were isolated using a Ficoll-Hypaque density gradient, selected by adherence to plastic, and stimulated with 100 ng/mL LPS for 48 hrs. RA fibroblast-like synoviocyte were isolated from remnant pannus obtained at knee arthroplasty. Pannus was minced, digested at 37° C. for 75 minutes with 1 mg/ml collagenase I, 0.1 mg/ml DNase I, and 0.015 mg/ml hyaluronidase, grown in RPMI and after 4-8 passages grown to confluence and stimulated with 25 ng/ml PDGF-BB (Sigma) for 10 minutes.

G. Mast cell stimulation. For immunoblotting analysis, C1.MC/57.1 mast cells were serum-starved for 6-8 hrs, pre-incubated imatinib for 2 hr, and stimulated for 10 min with 100 ng/mL stem cell factor (SCF, Peprotech), and lysates generated. For cytokine analysis, C1.MC/57.1 cells were preincubated with imatinib, stimulated with 100 ng/mL SCF for 96 hrs, and supernatants harvested for cytokine analysis.

H. Macrophage isolation and stimulation. Resident peritoneal macrophage were isolated from DBA/1 mice by i.p. injection and withdrawal of 5-7 ml of RPMI media, adherent macrophage cultured overnight, pre-treated with imatinib for 2 hrs, stimulated with 100 ng/mL M-CSF (Chemicon International) for 10 minutes, and lysates generated. For morphology studies, macrophages with stimulated for 72 hours in the presence or absence of 100 ng/ml M-CSF and imatinib.

I. B cell isolation and stimulation. B cells were isolated from naïve DBA/1 mouse spleens by negative selection with MACS beads (Miltenyi Biotec). B cells were shown to be pure by staining with the B cell marker CD19 and analyzing by flow cytometry. Isolated B cells were stimulated for 72 hours with μ-specific anti-IgM F(ab′)₂ (50 μg/ml; MP Biomedicals) or LPS (5 μg/ml; Sigma-Aldrich). To measure B cell proliferation, B cells were pulsed with 1 μCi [³H]thymidine (ICN Pharmaceuticals) for the final 18 hours of the stimulation and a betaplate scintillation counter (Perkin Elmer) was used to quantitate incorporated radioactivity.

J. T-cell stimulation. Splenocytes from anti-CII TCR transgenic mice were stimulated for 72 hrs with 0-40 μg/ml CII (Chondrex) and [³H]TdR (ICN Pharmaceuticals) added for the final 18 hrs of culture; supernatants were analyzed for cytokines.

K. Immunoblotting. Lysates were generated from stimulated peritoneal macrophage, C1.MC/57.1 mast cells, or fibroblast-like synoviocyte (lysis buffer: 1% NP-40, 0.1% SDS, 0.5% SDS, 10 mM EDTA, Halt protease inhibitor cocktail (Pierce) and phosphatase inhibitor cocktail 2 (Sigma)). Lysates were separated on 7.5% SDS-PAGE gels (Bio-Rad), transferred to PVDF membranes, probed with primary and secondary antibodies, and signal detected with SuperSignal West Pico Chemiluminescent Substrate (Pierce).

L. Reverse phase protein lysate arrays. C1.MC/57.1 mast cells were serum starved for 6 hrs, stimulated and lysed in an equal volume of 2× lysis buffer (100 mM Tris-HCl pH 6.8, 10 mM EDTA, 4% SDS, 10% glycerol, 2% 2-mercaptoethanol, 2% phosphatase inhibitor cocktail 2 (Sigma), and protease inhibitor cocktail (Pierce)). Stimulated peritoneal macrophage and fibroblast-like synoviocyte were lysed per above. As previously described (Chan, S. M., et al., Nat Med 10:1390-1396, (2004)), lysates were printed on FAST slides (Schleicher & Schuell), arrays probed with phospho-specific antibodies, followed by a horseradish peroxidase-conjugated anti-rabbit IgG antibody (Jackson Immunoresearch), followed by Bio-Rad Amplification Reagent and Opti-4CN Substrate (Bio-Rad), bound biotin detected with Cy3-streptavidin, slides scanned with a GenePix 4000B microarray scanner (Molecular Devices), and feature fluorescence intensities quantified with GenePix 5.0 Pro. Presented values (FIG. 4G and FIG. 5C) represent anti-protein tyrosine kinase antibody signal (Cy3) normalized to levels in unstimulated cells.

M. Synovial array analysis. As described (Robinson, W. H., et al., Nat Med 8:295-301, (2002); Hueber, W., et al., Arthritis Rheum 52:2645-2655, (2005)), 500+ peptides and proteins representing autoantigens were printed on SuperEpoxy slides (TeleChem), arrays were incubated with 1:150 dilutions of mouse sera, followed by Cy3-labeled anti-mouse IgG/M antibody (Jackson Immunoresearch), and scanned using a GenePix 4000B scanner. Median feature intensities for each antigen were calculated from the 4-8 duplicate features representing each antigen.

N. In vitro c-Kit phophosphorylation assay. Recombinant c-Kit was pre-incubated with small molecule inhibitors at various concentrations, ATP and substrate were added to initiate the phosphorylation reaction, the reactions stopped after 30 minutes, anti-phospho-tyrosine staining performed to detect phospho-c-Kit, and time-resolved fluorescence used to quantitate c-Kit phosphorylation (HTScan c-Kit Kinase Assay, Cell Signaling).

O. Immunohistochemistry of rheumatoid arthritis synovium. Synovium obtained from a patient with chronic RA at the time of knee replacement was fixed, paraffin embedded, and sections stained with monoclonal or polyclonal antibodies specific for c-Fms, PDGFRα, or PDGFRβ, or the corresponding isotype control antibodies, followed by HRP-based detection (Vector Labs).

P. Flow cytometric identification of mast cell, fibroblast like synoviocyte (FLS), and synovial macrophage populations derived from human RA synovial tissue. Remnant human knee synovium was obtained from an RA patient at the time of arthroplasty, and single cell suspensions generated by enzymatic digestion with Type IV collagenase. The resulting cell suspensions were stained with fluorochrome-conjugated antibodies specific for cell surface markers of hematopoietic cells (CD45), FLS (CD90), mast cells (c-Kit), and synovial macrophages (CD14), along with co-staining with an isotype matched control and anti-MHC class I antibodies. The presented plots represent the MHC class I positive cell populations.

Q. Cytokine analysis. Cytokine analysis was performed using the Beadlyte® Human or Mouse Multi-Cytokine Detection System (Chemicon International) and the Luminex 100 System (Luminex Corporation).

R. Statistical analysis. Visual arthritis scores, paw thicknesses and histology scores were compared by the Mann-Whitney U test using the GraphPad InStat Version 3.0 (GraphPad). Differences in CIA were determined by the Fisher test using the Analyse-it plug-in (Analyse-it software) for Excel (Microsoft). Cytokine level comparisons were performed using unpaired T tests (GraphPad). Significance Analysis of Microarrays (SAM; Tusher, V. G., et al., Proc Natl Acad Sci USA 98:5116-5121, (2001)) and Cluster and TreeView software (Eisen, M. B., et al., Proc Nat Acad Sci USA 95:14863-14868, (1998)) were used to analyze and display array data.

### Example 1

### Prevention of Collagen-Induced Arthritis with Imatinib

The ability of imatinib to prevent autoimmune arthritis in the collagen induced arthritis (CIA) model was evaluated as follows: CIA was induced by injecting DBA/1 mice with bovine type II collagen (CII) emulsified in CFA, followed by boosting 21 days later with CII emulsified in incomplete Freund's adjuvant (IFA).

DBA/1 mice (Jackson Laboratory) were administered phosphate buffered saline (n=15), 33 mg/kg imatinib (n=15), or 100 mg/kg imatinib (n=14) orally twice-daily starting one day prior to induction of CIA, based on the published pharmacokinetic profiles of imatinib metabolism in mice and humans (Druker, 2001; Buchdunger, 2001; Wolff, N. C., et al., Clin Cancer Res 10:3528-3534, (2004)). Imatinib is metabolized more rapidly in mice than in humans, and twice-daily oral dosing of mice with 100 mg/kg imatinib exhibits a similar pharmacokinetic profile as a mid-range dose of 400 mg once-daily in humans. This dosing regimen for mice and humans results in mean peak and trough plasma levels of 4.6-6 μM and 1-1.5 μM, respectively (Druker, 2001; Wolff, 2004).

For the CIA prevention studies, oral administration of imatinib was initiated 1 day prior to induction of CIA. Mice treated with either 33 or 100 mg/kg imatinib displayed significant reductions in the severity of CIA based on reduced paw swelling, erythema and joint rigidity as assessed by the mean visual arthritis score, as shown in FIG. 1A, and reduced mean paw thickness based on caliper measurements, as shown in FIG. 1B (p<0.01 by Mann-Whitney for both the 33 and 100 mg/kg groups after day 38 following primary immunization).

The incidence of arthritis at the termination of the experiment (day 49) and the mean weights of mice in each group were also measured, and the results are shown in FIGS. 1C and 1D. Imatinib also reduced the incidence of CIA, as seen in FIG. 1C. The therapeutic effects of imatinib demonstrated a trend towards dose-dependence (FIGS. 1A-1C). These results are representative of 3 independent experiments. There was no apparent toxicity or weight loss in mice receiving imatinib, as seen in FIG. 1D.

### Example 2

### Treatment of Collagen Induced Arthritis with Imatinib

The ability of imatinib to treat established autoimmune arthritis in the collagen induced arthritis (CIA) model was evaluated as follows. DBA/1 mice with established clinical arthritis (average visual score of 4, CIA model as described in Example 1) were randomized and treated with 33 or 100 mg/kg imatinib or PBS. Progression of established arthritis was assessed by both a visual scoring system and mean paw thickness based on caliper measurements. The results, shown in FIGS. 2A-2B, show that both the 33 and 100 mg/kg dose levels of imatinib inhibited the progression of established arthritis as assessed by both the visual scoring system and mean paw thickness (p<0.05 after 10 days following the initiation of treatment; values are mean ± SEM. *P<0.05, **P<0.01 compared with PBS-treated mice).

Histopathologic analysis was performed on hind paws harvested from mice with CIA receiving imatinib or PBS in the prevention (Example 1) and treatment studies. Representative images of H&E-stained joint tissue sections from imatinib and PBS treated mice in the CIA prevention studies are presented in FIGS. 3A-3C. Histopathologic evaluation by an investigator blinded to treatment group demonstrated that imatinib resulted in statistically significant reductions in synovitis, pannus, and erosion scores in both the CIA prevention study, as seen in FIGS. 3D-3F (p<0.01 by Mann-Whitney for synovitis and erosion scores, p<0.05 for pannus scores; PBS n=8, imatinib 33 mg/kg n=8, imatinib 100 mg/kg n=8) and in the study looking at treatment of established CIA, as seen in FIGS. 3G-3I (p<0.05 for synovitis, pannus and erosions scores; PBS n=8, imatinib 33 mg/kg n=8, imatinib 100 mg/kg n=8). Thus, imatinib was effective at both preventing and treating established CIA based on clinical and histopathologic analyses.

### Example 3

### Effect of Imatinib on Mast Cell Production of Cytokines and Signaling

A. Presence of Mast Cells in Inflamed CIA Synovial Tissue

To confirm prior observations that mast cells are present in joints derived from mice with CIA (Kakizoe, E., et al., Inflamm Res 48:318-324, (1999)), sections of CIA joints were stained with toluidine blue. Toluidine blue is a metachromatic dye that stains the strongly sulphated acid mucopolysaccharide (heparin) content of mast cell granules. Toluidine blue staining revealed significant numbers of mast cells in inflamed CIA synovial tissue, as seen in FIG. 4A. Mast cells present in the densely inflamed CIA synovial tissue are indicated by arrows in FIG. 4A (B=bone, JS=joint space. Original magnification 200×).

B. Inhibition of Mast Cell Production of Proinflammatory Cytokines with Imatinib

The effects of imatinib on activation of the cloned murine mast cell line C1.MC/57.1 (Young, J. D., et al., Proc Natl Acad Sci USA 84:9175-9179, (1987)) was evaluated as follows. C1.MC/57.1 mast cells expand in a growth factor-independent fashion, and although growth does not depend on stem cell factor (SCF), C1.MC/57.1 mast cells are responsive to SCF by secreting cytokines (Furuta, G. T., et al., Blood 92:1055-1061, (1998); Tsai, M., et al., FASEB J. 10:A1253, (1996)). C1.MC/57.1 mast cells were stimulated with 100 ng/mL SCF for 48 hrs in the presence of 0-5 μM imatinib, and cytokine analysis was performed on culture supernatants using a bead-based cytokine assay. As seen in FIGS. 4B-4D, imatinib at 1 μM and 5 μM dramatically reduced mast cell production of TNFα, GM-CSF, and IL-6 to levels similar to those in the unstimulated cell populations. Values are mean ± SEM *P<0.05, **P<0.01 compared with stimulated cells without imatinib.

C. Inhibition of Mast Cell Signaling by Stem Cell Factor with Imatinib

To characterize tyrosine kinase activation states and signal transduction pathways modulated by imatinib, immunoblot and reverse phase protein (RPP) lysate array (Chan, S. M., et al., Nat Med 10:1390-1396, (2004)) analyses were performed. Mast cells were pre-treated with 0-5 μM imatinib and stimulated with SCF for 10 minutes. Lysates were generated for immunoblotting and RPP lysate arrays. As seen in FIGS. 4E-4F, immunoblotting demonstrated that imatinib potently inhibited SCF-induced phosphorylation of c-Kit (FIG. 4E), with a corresponding reduction in phosphorylation of downstream Akt (Ser 473) (FIG. 4F).

RPP arrays were generated by printing cellular lysates on nitrocellulose-coated microscope slides, followed by incubation with phospho-specific antibodies and fluorescence-based detection of antibody binding to ascertain the activation of protein tyrosine kinases in the MAPK family and other pathways. As seen in FIG. 4G, RPP array analysis revealed that 1 and 5 μM imatinib inhibited SCF-induced activation of diverse protein tyrosine kinases downstream of c-Kit, including members of MAPK pathways including ERK, JNK, and p38. Imatinib also prevented phosphorylation of the signaling molecules Akt (Ser 473 and Thr 308), p70S6K, and Raf (FIG. 4G). Imatinib-mediated inhibition of c-Kit and Akt phosphorylation (Ser 473) was also observed in immunoblotting analysis (FIG. 4E), which serves as validation for RPP array results. Together, the immunoblots and RPP array data demonstrate that imatinib potently inhibits SCF-induced activation of MAPK and other pathways that mediate mast cell activation and pro-inflammatory cytokine production.

Mast cells influence both innate and adaptive immunity (Galli, S. J., et al., Nat Immunol 6:135-142, 2005), are present in rheumatoid synovial tissues, and may play an important role in the pathogenesis of RA (Woolley, D. E., 2003, N Engl J Med 348:1709-1711; Woolley, D. E. et al., 2000, Arthritis Res 2:65-74; Benoist, C., et al. Nature 420:875-878; Lee, D. M., et al. 2002, Science 297:1689-1692). Mast cells constitute approximately 5% of all cells in the hyperplastic synovial lining (known as the pannus tissue) in rheumatoid arthritis, and mast cells are also present in psoriatic skin lesions, multiple sclerosis brain plaques, and Crohn's inflamed bowel tissue. Mast cell granules contain abundant TNFα, IL-6, bradykinin, and a variety of proteases and other inflammatory and vascular permeability mediators (Juurikivi, A., et al. 2005, Ann Rheum Dis 64:1126-1131). c-Kit is a receptor tyrosine kinase for which stem cell factor (SCF) is the ligand. SCF-binding induces c-Kit phosphorylation and downstream activation of MAPK pathways in mast cells, resulting in release and production of TNFα, IL-6, and many other inflammatory mediators. Further, mast cell deficient mice exhibit less severe arthritis in models of RA and less severe demyelination in models of multiple sclerosis. Together these data suggest that mast cell production and release of TNFα, IL-6 and other mediators that promote vascular permeability and inflammation could contribute to the pathogenesis of rheumatoid arthritis, multiple sclerosis, Crohn's, psoriasis and a variety of other inflammatory and autoimmune conditions. Thus, inhibition of mast cell activation and pro-inflammatory mediator release by imatinib or other tyrosine kinase inhibitors that inhibit c-Kit could provide benefit in RA and other inflammatory diseases.

### Example 4

### Effect of Imatinib on Monocyte Lineage Cell Differentiation and Function

M-CSF, which binds and signals through the receptor tyrosine kinase Fms, is present in RA synovial tissue and has been shown to exacerbate CIA (Campbell, I. K., et al., J Leukoc Biol 68:144-150, (2000). To determine whether imatinib affects M-CSF-mediated signal transduction in macrophage, immunoblotting and RPP arrays were applied to characterize lysates generated from resident peritoneal macrophage. Resident peritoneal macrophage were isolated from DBA/1 mice, pre-treated with imatinib, and stimulated with 100 ng/mL M-CSF for 10 minutes and lysates generated for analysis. The immunoblots showed that 1 and 5 μM imatinib inhibited M-CSF-induced phosphorylation of c-Fms, while levels of total c-Fms were similar in all samples, as shown in FIG. 5A. The downstream signaling molecule Akt (Ser 473) also exhibited reduced phosphorylation in imatinib pre-treated macrophage, as seen in FIG. 5B.

RPP array analysis of M-CSF-stimulated macrophage lysates demonstrated that imatinib blocked phosphorylation of protein tyrosine kinases in the MAPK family and other pathways downstream of c-Fms, including Akt (Ser 473 and Thr 308), ERK, STAT3, JNK, P70S6K and p38, as seen in FIG. 5C. Thus, the immunoblots and RPP array data demonstrate that imatinib potently inhibits M-CSF-induced macrophage activation through c-Fms.

As shown in FIG. 5D, RA-derived synovial fluid mononuclear cells that adhered to plastic exhibited the morphology of monocytes when cultured in culture medium alone for 72 hours. Following stimulation with M-CSF for 72 hours, these cells differentiated into macrophages, with classical macrophage morphologic characteristics that include multipolar process extension, heterogeneous cytoplasmic vacuoles and inclusions, as seen in FIG. 5E. Cells that were co-incubated with 5 μM imatinib and M-CSF displayed a morphology similar to unstimulated cells, shown in FIG. 5F. Thus, imatinib blocked the differentiation of synovial fluid monocytes from human RA patients into macrophages.

### Example 5

### Effect of Imatinib on B Cell Responses

A. Imatinib Inhibits B Cell Proliferation and Immunoglobulin Production

B cells from naïve DBA/1 mice were isolated from whole splenocytes and their purity verified by flow cytometry, shown in FIG. 6A. Post-isolation B cells were stimulated for 72 hours with anti-IgM (50 μg/ml) or LPS (5 μg/ml) in the presence or absence of 1-10 μM imatinib. B cell proliferation with anti-IgM was inhibited by imatinib concentrations as low as 5 μM (p<0.001), as seen in FIG. 5B. LPS-stimulated B cells demonstrated reduced proliferation in a dose-dependent fashion (p<0.001 for 1 μM and higher concentrations), shown in FIG. 5C. Further, as seen in FIG. 5D, IgM production by LPS-stimulated B cells was mildly reduced by imatinib at a concentration of 1 μM, and exhibited the most significant reduction at 10 μM.

B. Imatinib Reduces Epitope Spreading of Autoreactive B Cell Responses

Synovial array profiling of serum autoantibodies derived from mice with CIA treated with PBS (n=7) or 100 mg/kg imatinib (n=7) (day 49) was done. A robotic microarrayer was used to produce synovial arrays containing a spectrum of proteins and peptides representing candidate autoantigens in RA and CIA (Robinson, W. H., et al., Nat Med 8:295-301, (2002); Hueber, W., et al., Arthritis Rheum 52:2645-2655, (2005)). Arrays were incubated with 1:150 dilutions of sera, autoantibody binding detected with Cy3-labeled anti-mouse IgG/M, and arrays scanned and median fluorescence for each antigen determined. Synovial array analysis demonstrated that in vivo treatment of CIA mice with imatinib reduced expansion of autoreactive B cell responses to native epitopes representing glycoprotein 39 (gp39), clusterin, histone 2B (H2B), hnRNPB1 and vimentin as well as to citrullinated epitopes derived from filaggrin (cyc-filaggrin, cfc8 and CCP cyc Ala-12) and clusterin, as seen in FIG. 6E. In FIG. 6E, significance analysis of microarrays (SAM) was applied to identify antigen features with statistically increased reactivity in PBS as compared to imatinib treated mice (false discovery rate (FDR)=0.06). Cluster and TreeView software was applied to order and display the array reactivity as a heatmap. Samples from vehicle (phosphate-buffered saline, PBS) treated control mice cluster on the left side of the heatmap and exhibit high antibody reactivity against multiple synovial proteins, while the imatinib treated mice cluster on the right side and exhibit reduced autoantibody titers.

### Example 6

### Effect of Imatinib on T Cell Responses

The impact of imatinib on T cells expressing a transgenic TCR specific for CII peptide 257-72 was investigated (Osman, G. E., et al., Int Immunol 10:1613-1622, (1998)). Splenocytes derived from a mouse expressing a transgene encoding a CII-specific TCR were stimulated with 0-40 μg/mL heat-denatured whole CII in the presence of 0-10 μM imatinib. ³H-thymidine incorporation was used to measure proliferation of CII-specific T cells. When stimulated in the presence of 1 μM or 3.3 μM imatinib, CII-specific T cells proliferated robustly to heat-denatured whole CII, while 10 μM imatinib (exceeding the 14.6 μM blood level achieved by a mid-range human dose (Druker, 2001; Demetri 2002) potently inhibited proliferation, as seen in FIG. 7A.

A moderate reduction in production of proinflammatory IFN-γ and TNFα by CII-stimulated (20 μg/mL) TCR transgenic T cells was observed at 3.3 μM imatinib, while supra-therapeutic 10 μM imatinib further reduced production of the immunomodulatory cytokines IFN-gamma, IL-4, and TNFα as seen in FIGS. 7B-7D. Anti-CII T cell production of IL-2 in response to CII was not significantly reduced at any of the imatinib concentrations tested (FIG. 7E). Values are mean ± SEM. *P <0.05, **P<0.01 compared with stimulated cells without imatinib. The T cells appeared viable in these experiments and 10 μM imatinib did not upregulate early or late apoptosis markers Annexin V or propidium iodide by flow cytometry after 5 hours (Table 3) or 24 hours (Table 4) of incubation. Table 3 presents results from analysis of splenocytes from anti-CII TCR transgenic mice that were stimulated with CII and then stained after 5 hours with mAbs against the pan T cell marker CD3, propidium iodide, and Annexin V to determine early apoptosis (PI⁻ Annexin V⁻) as well as late apoptosis or cell death (PI⁺ Annexin V⁻) using flow cytometry. Table 4 shows CII-stimulated splenocytes that were stained after 24 hours with anti-CD4 antibody, propidium iodide, and Annexin V following which flow cytometry was performed. The data presented in Tables 3 and 4 show that imatinib treatment did not significantly alter staining of these T cells with Annexin V (stains early apoptotic cells) or propidium iodide (stains dead cells) based on flow cytometry analysis, demonstrating that imatinib did not induce apoptosis or death in these cells. Together, these data suggest that imatnib inhibits T cell responses via inhibition of Lck.

TABLE 3
Imatinib did not cause apoptosis or death
of anti-CII T cells after 5 hours.

| Imatinib | CII | Live cells | Early apoptosis | Late apoptosis |
|---|---|---|---|---|
| (μM | (μg/ml) | (PI− AnnV−) | (PI− AnnV+) | (PI+ AnnV+) |
| 0 | 0 | 93.1% | 5.6% | 1.2% |
| 1 | 0 | 93.8% | 5.2% | 0.9% |
| 2.5 | 0 | 93.3% | 5.2% | 1.3% |
| 5 | 0 | 92.0% | 7.0% | 1.0% |
| 10 | 0 | 92.7% | 6.3% | 0.9% |
| 0 | 10 | 94.0% | 5.1% | 0.9% |
| 1 | 10 | 92.7% | 6.4% | 0.8% |
| 2.5 | 10 | 91.2% | 7.7% | 1.0% |
| 5 | 10 | 90.5% | 8.5% | 0.9% |
| 10 | 10 | 91.0% | 7.8% | 1.1% |
| 0 | 40 | 94.0% | 5.2% | 0.7% |
| 1 | 40 | 92.9% | 6.3% | 0.7% |
| 2.5 | 40 | 92.3% | 7.0% | 0.7% |
| 5 | 40 | 91.8% | 6.9% | 1.3% |
| 10 | 40 | 91.6% | 7.3% | 0.9% |

TABLE 4
Imatinib did not cause apoptosis or death
of anti-CII T cells after 24 hours.

| Imatinib | CII | Live cells | Early apoptosis | Late apoptosis |
|---|---|---|---|---|
| (μM) | (μg/ml) | (PI− AnnV−) | (PI− AnnV+) | (PI+ AnnV+) |
| 0 | 0 | 95.0% | 4.3% | 0.7% |
| 1 | 0 | 95.5% | 3.8% | 0.6% |
| 2.5 | 0 | 94.6% | 4.3% | 1.0% |
| 5 | 0 | 93.3% | 5.9% | 0.8% |
| 10 | 0 | 92.9% | 6.1% | 0.9% |
| 0 | 10 | 96.2% | 3.4% | 0.4% |
| 1 | 10 | 96.1% | 3.4% | 0.5% |
| 2.5 | 10 | 94.7% | 4.7% | 0.5% |
| 5 | 10 | 94.0% | 5.3% | 0.7% |
| 10 | 10 | 92.4% | 6.6% | 0.9% |
| 0 | 40 | 96.0% | 3.2% | 0.7% |
| 1 | 40 | 94.3% | 4.6% | 1.1% |
| 2.5 | 40 | 94.2% | 4.9% | 0.9% |
| 5 | 40 | 93.6% | 5.2% | 1.2% |
| 10 | 40 | 93.7% | 6.2% | 0.9% |

### Example 7

### Effect of Imatinib on Cytokine Production and Fibroblast PDGFR in RA Explants

Mononuclear cells were isolated from synovial fluid derived from RA patients. Because M-CSF induces mononuclear cell maturation but not TNFα production (Pixley, F. J., and Stanley, E. R., Trends Cell Biol 14:628-638, (2004)), LPS was used to stimulate synovial fluid mononuclear cells to produce TNFα (Wolf, A. M., et al., Proc Natl Acad Sci USA 102:13622-13627, (2005); Dewar, A. L., et al., Immunol Cell Biol 83:48-56, (2005)), the archetypal pro-inflammatory cytokine in RA. Synovial fluid mononuclear cells were isolated and stimulated in vitro with 100 ng/mL LPS for 48 hrs in the presence of 0-8 μM imatinib. Mononuclear cell supernatants were harvested and a bead-based cytokine assay utilized to quantify TNFα, IL-12(p40) and IL-1α. As seen in FIGS. 8A-8C, bead-based cytokine analysis of culture supernatants demonstrated reductions in production of pro-inflammatory cytokines including TNFα (FIG. 8A) and to a lesser degree IL-12 (FIG. 8B). Imatinib did not inhibit LPS-induced production of IL-1α, indicating that imatinib did not affect synovial fluid mononuclear cell viability (FIG. 8C). (Values are mean ± SEM. *P<0.05, **P<0.01 compared with stimulated cells without imatinib).

Fibroblast-like synoviocyte (FLS) were isolated from pannus derived from a human RA patient at the time of knee arthroplasty. Cultured FLS were preincubated with 0-5 μM imatinib followed by stimulation with 25 ng/mL PDGF-BB for 10 minutes. Lysates were generated and probed by immunoblotting for p-PDGFRβ and total PDGFRβ, and for p-Akt and total Akt. Imatinib at 0.5 and 5 μM potently inhibited phosphorylation of PDGFR, as well as the downstream signaling molecule Akt, as seen in FIGS. 8D and 8E.

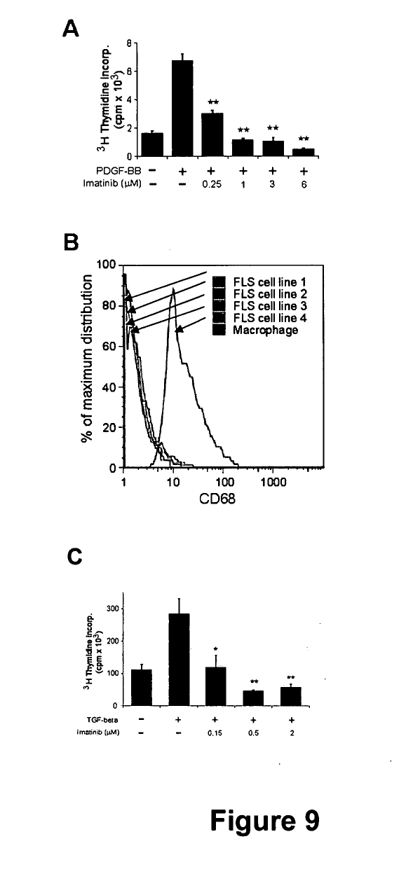

NIH-3T3 fibroblasts were stimulated with 20 ng/mL PDGF-BB, a stimulus previously demonstrated to induce proliferation of NIH-3T3 cells (Osman, 1998), in the presence of 0-8 μM imatinib and proliferation measured by ³H-thymidine incorporation. Proliferation was measured by ³H-thymidine incorporation. As seen in FIG. 9A, imatinib concentrations as low as 0.25 μM inhibited PDGF-BB-induced proliferation of fibroblast-like synoviocyte (FLS) cells derived from a human RA patient (p<0.01). Values are mean ± SEM. **P<0.01 compared with stimulated cells without imatinib. These results are representative of results obtained from FLS derived from four different rheumatoid arthritis patients. The data in FIG. 9B confirms the absence of contaminating macrophage by flow cytometric analysis with anti-CD68 antibody (peritoneal cells=peritoneal macrophage for which CD68 is a lineage marker).

In addition, in RA synovial fibroblasts express platelet-derived growth factor receptor (PDGFR, referring to PDGFRα and PDGFRβ collectively)) and proliferate in response to a variety of platelet-derived growth factor (PDGF) ligands. Both PDGFR and its ligands are over-expressed in rheumatoid arthritis synovial tissue, and PDGF is a potent stimulant of synovial fibroblast proliferation (Cheon, H., et al., Scand J Immunol 60:455-462, (2004); Watanabe, N., et al., Biochem Biophys Res Commun 294:1121-1129, (2002); Remmers, E. F., et al., J Rheumatol 18:7-13, (1991)). Further, macrophage are believed to play a central role in producing pro-inflammatory cytokines such as TNFα in rheumatoid arthritis.

TGF-β is an important profibrotic pathway (Distler, J., et al., Arthritis Rheum 56:311-333 (2007)). TGF-β stimulation in fibroblasts signals through c-Abl (Wang, S., et al., FASEB J 19:1-11 (2005)), and FIG. 9C shows that imatinib inhibition of c-Abl potently blocks fibroblast proliferation. Thus, imatinib and other tyrosine kinase inhibitors that inhibit c-Abl may also inhibit synovial fibroblast (also known as fibroblast like synoviocytes, FLS) proliferation in RA. It is possible that co-inhibition of both PDGFR and Abl by imatinib or other tyrosine kinase inhibitors could synergize to inhibit proliferation of synovial fibroblasts or other aberrant cellar responses in autoimmune disease.

Substantial evidence exists that dysregulated responses that can include fibrotic-like responses occur in and contribute to the pathogenesis of many autoimmune and inflammatory diseases. In multiple sclerosis there is reactive gliosis (scaring) in areas of damaged white matter. The cytokine IL-6 plays an important role in gliosis (Woiciechowsky, C. et al. (2004) Med Sci Monit. 10:BR325-30). Further, fibroblast growth factor receptor (FGFR) and PDGFR are expressed on astrocytes and mediate astrocyte differentiation and proliferation to cause gliosis and scaring in autoimmune demylination (Cassina, P. et al. (2005) Neurochem. 93:38-46; Takamiya, Y. et al. (1986) Brain Res. 383:305-9; Yamada, H. et al. (2000) Am. J. Pathol. 156:477-87). In systemic lupus erythematosus glomerulonephritis and renal damage is characterized by glomerulosclerosis in severe disease (Kraft, S. W. et al, (2005) J. Am. Soc. Nephrol. 16:175-179). Autoimmune hepatitis and primary biliary cirrhosis are characterized by fibrosis of the liver parenchyma and biliary tree, respectively (Washington, M. K. (2007) Mod Pathol. 20 Suppl 1:S 15-30). Idiopathic pulmonary fibrosis is a fibrotic disease of the lung, and TGF-β mediated fibrosis plays a central role in the pathology of this disease (Ask, K. (2006) Proc. Am. Thorac. Soc. 3:389-93). Crohn's disease is characterized by fibrosis of the bowel wall and mesnetary, and fibrosis results in the bowel strictures that are characteristic of this disease (Sorrentino, D. (2007) Digestion. 75:22-4).

Thus, imatinib and other tyrosine kinase inhibitors that inhibit PDGFR or Abl could inhibit the proliferation of synovial fibroblasts and the proliferation of other fibroblast-like cells that contribute to the pathogenesis of RA, systemic sclerosis, Crohn's, multiple sclerosis, autoimmune hepatitis and other inflammatory diseases in which such responses contribute to pathogenesis.

### Example 8

### Effect of Imatinib on Multiple Sclerosis

A study was conducted to determine the ability of imatinib mesylate (imatinib) to prevent and treat experimental autoimmune encephalomyelitis (EAE), a mouse model of multiple sclerosis (MS). EAE was induced in C57B/6 mice by subcutaneous immunization with 100 ug/mouse myelin oligodendrocyte glycoprotein (MOG) peptide 35-55 emulsified in compete Freund's adjuvant (CFA) containing 2 mg/ml heat-killed mycobacterium tuberculosis H37Ra (Difco Laboratories, Detroit, Mich.). As part of the induction protocol, mice were also injected intravenously on the day of immunization and 48 hours later with 0.1 ml of 4 μg/mL Bordetella pertusis toxin. Severity of EAE was determined daily based on a standard scoring system: 1, tail weakness or paralysis; 2, hind leg weakness; 3, hind limb paralysis; 4, forelimb weakness or paralysis; and 5, moribund animals or death. Mice treated with 100 mg/kg imatinib twice daily demonstrated a delay in the onset and reduced severity of EAE compared to the PBS-vehicle control mice, as seen in FIG. 10 (values are mean ±s.e.m.). These data demonstrate that imatinib is also efficacious in treating a rodent model of multiple sclerosis.

### Example 9

### The Fms and PDGFR Inhibitor GW2580 for Treating Rheumatoid Arthritis

As documented above, imatinib significantly prevents the onset and severity of CIA. One likely mechanism by which imatinib may exhibit efficacy is due to inhibiting c-Fms on monocyte lineage cells. GW2580 was purchased from Calbiochem (Catalog #344036). GW2580 is highly potent for c-Fms, and in vitro FLS proliferation assays demonstrate that GW2580 also inhibits PDGFR at levels achieved by a standard murine dosing regimen (IC₅₀ 4.3 mM; FIG. 11B) (Conway, J., et al., Proc Natl Acad Sci USA 102:16078-16083, (2005)). In vitro c-Kit phosphorylation assays demonstrated that GW2580 did not inhibit c-Kit at concentrations achieved in standard murine dosing regiments (IC₅₀ 73.5 μM) (Conway, J., et al., Proc Natl Acad Sci USA 102:16078-16083, (2005)). As shown in FIGS. 12A and 12B, the c-Fms-specific small molecule tyrosine kinase inhibitor GW2580 was highly effective at treating established collagen-induced arthritis in mice, a rodent model for RA as assessed by both the mean arthritis score (A) and paw thickness (B). Fms plays a central role in the differentiation of immature monocytes into macrophage and osteoclasts, as well as in the priming macrophage to produce TNFα and other cytokines and the activation of osteoclasts (C). Monocyte differentiation assays were performed to further assess the ability of GW2580 and imatinib to inhibit differentiation of monocytes into macrophage. Human peripheral blood monocytic cells were stimulated with CSF-1 to induce differentiation into macrophage, and cells were co-incubated with varying concentrations of imatinib or GW2580. Both imatinib and GW2580 blocked M-CSF-induced differentiation of human blood monocytes to macrophages (FIG. 12D). Macrophages were counted based on morphologic features including cytoplasmic inclusions, multipolar process extension, and heterogeneous cytoplasmic vacuoles (% macrophage represents the % of total cells with morphologic features characteristic of macrophage). IC₅₀ data were generated for imatinib (0.97 μM) and GW2580 (0.01 μM) for inhibition of Fms, and GW2580 inhibited M-CSF-induced differentiation of monocytes to macrophages approximately 100 times more potently than imatinib (FIG. 12E). Thus, the GW2580 tyrosine kinase inhibitor, which is highly potent for Fms and also inhibits PDGFR, was highly effective at treating established collagen-induced arthritis.

The ability of a Fms and PDGFR inhibitor to treat autoimmune arthritis was unexpected, and provides new insights into the pathogenic mechanisms underlying RA. c-Fms (colony-stimulating factor-1 receptor (CSF-1R)) is expressed on cells of the monocyte lineage, and mediates monocyte differentiation into macrophage and osteoclasts. Fms also primes and activates macrophage to produce TNFα, other inflammatory cytokines, and to carry out other macrophage functions. Macrophages are professional antigen-presenting cells and are also effector cells that secrete TNFα when activated. TNFα plays a central role in synovitis and joint destruction in human rheumatoid arthritis (RA), psoriasis, psoriatic arthritis, inflammatory bowel diseases including Crohn's disease, ankylosing spondylitis and other autoimmune diseases. Macrophages are thought to be a primary source of TNFα production in these autoimmune diseases. Further, three biological agents that inhibit TNFα are approved by the U.S. Food and Drug Administration for the treatment of RA, Crohn's, and psoriasis, and have shown efficacy in treating psoriatic arthritis and ankylosing spondylitis. Osteoclasts play a central role in the breakdown of bone that results in bony erosions and destruction in RA, and likely in other inflammatory arthritidies such as psoriatic arthritis and ankylosing spondylitis.

Fms also plays a central role in the activation of osteoclasts to mediate bone erosions and destruction in these inflammatory arthritidies. Osteoclasts play a primary role in bone erosion and destruction in rheumatoid arthritis. Osteoclasts are multinucleated cells of the myeloid pathway whose primary function is bone resorbtion. In rheumatoid arthritis and collagen-induced arthritis, osteoclasts are found both within the bone and within synovial tissue at sites adjacent to bone (Schett, G., (2007), Arthritis Res Ther 9:203). Osteoclasts utilize enzymes and a proton pump to degrade bone matrix and absorb Ca⁺⁺, respectively (Teitelbaum et al, (2000), Science 289:1504-1508). Stimulation of myeloid precursors through c-Fms and RANKL induces differentiation of myeloid precursors into osteoclasts (Theill et al. (2002) Annu. Rev. Immunol. 20:795-823.). The RA synovium is theorized to accumulate osteoclasts based on the presence of monocytes and other myeloid osteoclast precursors combined with cells that provide signals (CSF-1, RANKL, IL-17) that simulate osteoclast formation (Schett, G. (2007) Arthritis Res. Ther. 9:203). Inhibition of c-Fms could ameliorate autoimmune arthritis and other autoimmune diseases by inhibiting the differentiation of myeloid precursors into macrophages and osteoclasts, as well as by inhibiting priming and activation of macrophages and osteoclasts.

It is likely that the benefit observed from GW2580, a highly potent Fms inhibitor that also inhibits PDGFR at concentrations achieved by murine dosing regimens, was due to concordant inhibition of: (i) myeloid lineage differentiation into TNFα producing macrophage and bone-eroding osteoclasts, and (ii) synovial fibroblast proliferation to form invasive pannus tissue. These data suggest that a tyrosine kinase inhibitor that inhibits Fms and/or PDGFR could provide efficacy in human RA.

### Example 10

### The PDGFR, FGFR and c-Kit Inhibitor CGP53716 Prevents Autoimmune Arthritis

An in vitro c-Kit phosphorylation assay (FIG. 11A) and in vitro FLS proliferation assay (FIG. 11B) demonstrated that the small molecule tyrosine kinase inhibitor CGP53716 potently inhibits both c-Kit activity in an in vitro kinase assay and PDGFbb-induced FLS proliferation. Specific inhibition of PDGFR, FGFR and c-Kit was highly effective at preventing the onset of and reducing the severity of CIA (FIG. 13A-13B). In addition to PDGFR, FGFR has been demonstrated to mediate proliferation of synovial fibroblasts (Malemud, C. J., (2007), Clin Chim Acta. 375(1-2):10-9.). Blocking PDGFR, FGFR and c-Kit with CGP53716 was nearly as effective as imatinib at preventing and treating CIA (FIG. 13C-13D).

### Example 11

### Expression of PDGFRα, PDGFRβ and c-Fms in Human Rheumatoid Arthritis Synovium

Immunohistochemistry was performed to characterize the expression of Fms and PDGFR in RA synovium (pannus). A high level of c-Fms protein was present at the surface of the rheumatoid synovium (FIG. 14A-14B), and lower levels in the underlying synovial tissue. PDGFRα was predominantly expressed deeper in synovial tissue (FIGS. 14C and 14D), while PDGFRβ was intensely expressed by a subset of cells near the synovial lining (FIGS. 14E and 14F). These results further implicated the involvement of these tyrosine kinases in the pathogenesis of RA.

### Example 12

### Mast Cells, Fibroblast-Like Synoviocyte (FLS), and Synovial Macrophage Populations in Human Rheumatoid Arthritis Synovial Tissue

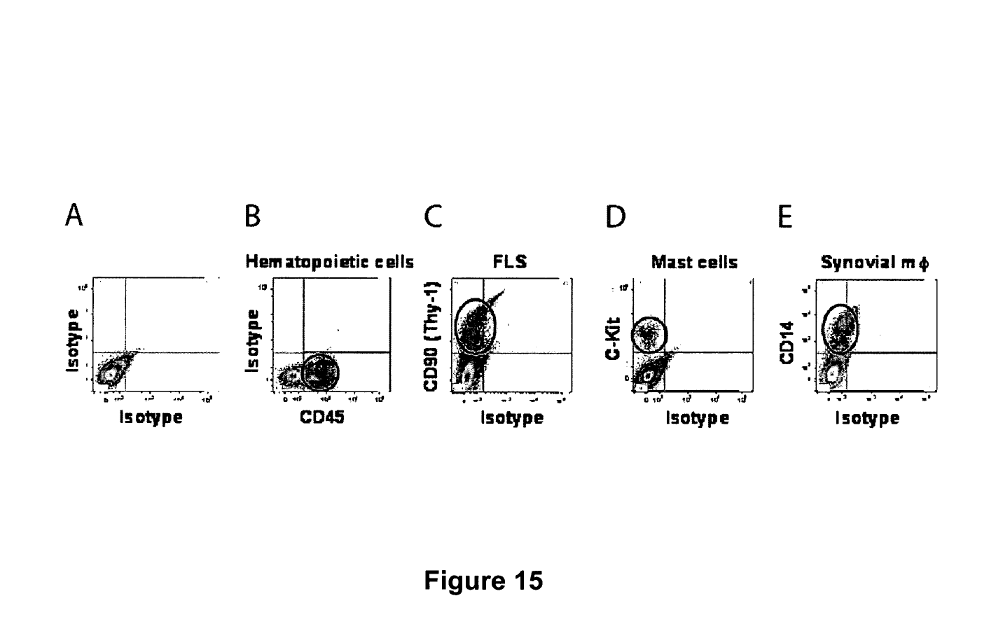

FIG. 15 presents results from flow cytometry analysis of fresh, uncultured cells isolated directly from RA synovial tissue. Individual stains demonstrate the detection of haematopoietic cells based on staining with anti-CD45, FLS by staining with anti-CD90, mast cells by staining with anti-c-Kit, and synovial macrophages by staining with anti-CD14. Flow cyotmetric analysis demonstrated distinct populations of fibroblasts-like synoviocytes (FLS), mast cells, and synovial macrophages in human RA synovial tissue. These results further implicated the involvement of these cell types in the pathogenesis of RA.

### Example 13

### Low-Dose Imatinib in Combination with Low-Dose Atorvastatin, Rosiglitazone, or Enoxaparin Treats RA in a Rodent Model

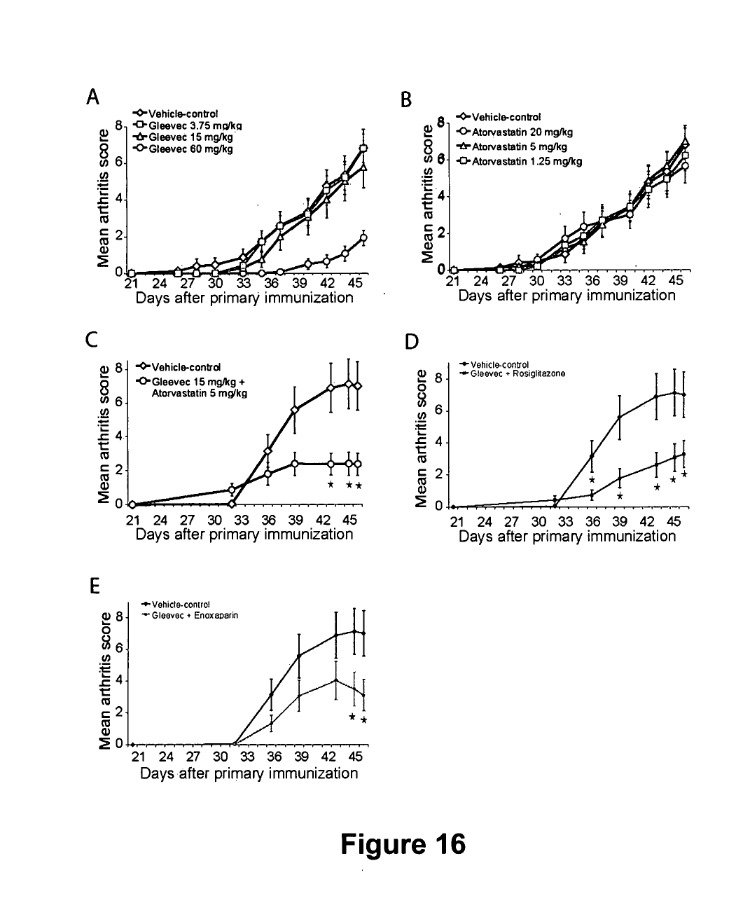

FIG. 16A shows a CIA efficacy titration curve of different concentrations of imatinib. It was observed that 15 mg/kg imatinib exhibits relatively little efficacy compared to the PBS vehicle-control. Rheumatoid arthritis patients have a higher risk of developing cardiovascular disease, and administering statin drugs such as atorvastatin is often indicated. Atorvastatin alone, at 1.25 to 20 mg/kg, was not effective at decreasing the clinical scores of CIA (FIG. 16B). However, when CIA mice were dosed with a combination of 15 mg/kg imatinib and 5 mg/kg atorvastatin, these mice developed significantly less severe disease than vehicle alone (FIG. 16C).

In addition, low-dose imatinib at 15 mg/kg in combination with low-dose rosiglitazone or low-dose enoxaparin was effective at preventing the onset and severity of CIA, as shown in FIGS. 16D and 16E. These results suggest that combination therapy of imatinib and other tyrosine kinase inhibitors with non-tyrosine kinase inhibitor therapies could provide increased benefit. Examples of existing and novel non-tyrosine therapies include the therapies tested in this example; small molecule anti-proliferative therapies (methotrexate, mycophenolate mofetil, imuran); anti-cytokine therapies (anti-TNF, anti-IL-6, anti-IL-1); inhibitors of immune cell trafficking (anti-VLA4 (Tysabri)); and anti-B cell therapies (rituximab (anti-CD20), anti-CD19); antigen-specific tolerizing therapies.

### Example 14

### c-Kit-Mutant Mice Develop Less Severe Antibody-Transfer Arthritis than Wild-Type Mice

Wsh mice are genetically modified to have a mutation that interferes with c-Kit signaling, and the result is that these mice are mast cell-deficient. As shown in FIG. 17A-17B, c-Kit-mutant mice were partially resistant to developing arthritis in the collagen antibody-induced arthritis model, compared to wild-type control mice that have normal c-Kit functions. These observations further support inhibition of c-Kit as a therapeutic approach in RA and other autoimmune diseases, but suggest that inhibition of Kit alone will likely be insufficient to treat autoimmune arthritis or other autoimmune diseases.

### Example 15

### Imatinib for the Treatment of Systemic Sclerosis

A 24-year old female with a three-year history of SSc presented with rapidly progressive cutaneous sclerosis, multiple digital ulcers (FIG. 18A, and increasing shortness of breath to the point that she was only able to walk half of a block. Pulmonary function tests showed a progressive decline in FVC to 48% predicted with a stable diffusion capacity of 62% predicted. HRCT scan of the chest demonstrated increased bibasilar ground glass opacities (FIG. 18C). A transthoracic echocardiogram (TTE) showed normal right and left ventricular function, a right ventricular systolic pressure (RVSP) of 21, and a small pericardial effusion. The patient declined cyclophosphamide therapy for her ILD, and was referred to our center for further treatment options.

The patient agreed to a trial of imatinib mesylate. Written informed consent was obtained to collect clinical information in the form of photographs and questionnaires, as well as skin and blood samples for molecular analyses. This study was approved by the local institutional review board. Prior to initiating therapy, the patient's modified Rodnan skin thickness score (mRSS) was 36 (scale 0-51). Her oral aperture measured 1.0 cm and her hand extension measurements were 13.0 cm on the left, and 10.2 cm on the right. She had nine digital ulcers and contractures at her elbows and left fourth digit. Her complete blood count, comprehensive metabolic panel, creatine kinase, and urinalysis were within normal limits. C-reactive protein (CRP) level was 2.8 mg/dL (normal <0.5 mg/dL). A skin biopsy demonstrated thickened, closely packed collagen bundles with an average dermal thickness of 2.81 mm (FIG. 18E).

After three months of imatinib mesylate at 100 mg twice daily, the patient reported softening of her skin, increased joint mobility, and decreased shortness of breath. Physical examination revealed a mRSS of 21 and four digital ulcers, with significant healing of five of the digital ulcer (FIG. 18B). CRP had normalized to 0.2 mg/dL and the patient had been able to taper her prednisone to 5 mg daily. HRCT showed resolution of the interstitial changes (FIG. 18D) and a repeat TTE showed no evidence of a pericardial effusion. A repeat skin biopsy showed more widely spaced, thinner collagen bundles with an average dermal thickness of 2.31 mm (FIG. 18F).

The patient subsequently increased the dose of imatinib by 50 mg/day every 4 weeks to a dose of 350 mg/day. After 6 months of imatinib therapy, the patient was able to return to work and reported walking 4 miles with only mild dyspnea. The patient's Health Assessment Questionnaire disability index (HAQ-DI) and scleroderma-specific visual analogue scale scores both improved substantially after 6 months of therapy. She had tapered off of her prednisone and reported substantial improvement in her joint pain. Physical examination demonstrated a mRSS of 18, and improvement in the hand extension measurement of her right hand to 14.5 cm. Her oral aperture had increased to 1.5 cm and she only had 2 remaining digital ulcers.

Imatinib likely provided benefit in this patient with systemic sclerosis by inhibiting: (i) PDGFR-mediated and Abl-mediated proliferation and collagen production by dermal fibroblasts; (ii) Fms-mediated monocyte differentiation into macrophage, and priming of macrophage to produce TNFα and other inflammatory cytokines; (iii) possibly Kit-mediated mast cell inflammatory mediator release; and (iv) possibly T and B cell function via Lck and Abl, respectively. Thus, simultaneous inhibition of multiple tyrosine kinases and the pathogenic cellular responses they mediate is likely central to the efficacy of imatinib observed in this patient with systemic sclerosis.

### Example 16

### Small Molecule Tyrosine Kinase Inhibitors and Selection of Candidates for Clinical Development for Inflammatory and Autoimmune Diseases

The chemical structures of small molecule tyrosine kinase inhibitors are presented in FIGS. 19 and 20. FIG. 19 presents the chemical structures of SU9518 (inhibits PDGFR and FGFR), GW2580 (inhibits Fms and PDGFR), CGP53716 (inhibits PDGFR, FGFR and Kit), and PD166326 (inhibits Kit and Abl) (Table 5). FIG. 20 presents the chemical structures of the FDA-approved tyrosine kinase inhibitors (Table 6), all of which were FDA-approved for the treatment of malignancies that arise from mutations in tyrosine kinases. Table 7 presents a list of additional tyrosine kinase inhibitors in clinical development, primarily to treat malignancies, that could potentially provide benefit in autoimmune and other inflammatory conditions. The IC₅₀ is the concentration of the kinase inhibitor at which 50% of kinase activity is inhibited. Additional tyrosine kinase inhibitors are in pre-clinical development, or have yet to be discovered.

TABLE 5
Initial small molecule tyrosine kinase inhibitors being investigated in rodent models of inflammatory and autoimmune diseases.

| | Inhibitory Profile (IC50, μM) | | | | | | | |
|---|---|---|---|---|---|---|---|---|
| Inhibitor | PDGFR | FGFR | Kit | Fms | Lck | Abl | Murine dosing | References |
| Imatinib | 0.1 | | 0.1 | 1.4 | 1-10 | 0.25 | 100 mg/kg/2×/d oral | Buchdunger, 2004. Clin Cancer Res |
| | | | | | | | | 10: 3528-3534. |
| SU9518 | 0.05 | 4.4 | | | | | 100 mg/kg/wk SQ or | Abdollahi, 2005. Inhibition of |
| | | | | | | | 50 mg/kg/d oral | platelet-derived growth factor |
| | | | | | | | | signaling attenuates pulmonary |
| | | | | | | | | fibrosis. J Exp Med 201: 925-935. |
| | | | | | | | | 58; Yamasaki, 2001. Circ Res |
| | | | | | | | | 88: 630-636. |
| GW2580 | 4.3 | | >10 | 0.03 | >10 | | 80 mg/kg/d (40) | Conway, 2005. Proc Natl Acad Sci |
| | | | | | | | | USA 102: 16078-16083; FIG. 11 |
| PD166326 | | | 0.025 | | | 0.01 | 50 mg/kg/2×/d oral | Wolff, 2005 Blood 105: 3995-4003. |
| CGP53716 | <1 | 1.1 | <1 | | | | 50 mg/kg/d oral | Myllamiemi, 1997. Faseb J 11: 1119-1126. |
| | | | | | | | | Buchdunger, 1995. Proc Natl Acad |
| | | | | | | | | Sci USA 92: 2558-2562. |

TABLE 6
Inhibitory Profiles of FDA-Approved Tyrosine Kinase Inhibitors

| | Inhibitory profile (with IC50s when available) | | | | | | | |
|---|---|---|---|---|---|---|---|---|
| Compound | Fms | PDGFR | Kit | Abl | Lck | VEGFR1 | VEGFR2 | VEGFR3 |
| Imatinib | 1 | 0.1 | 0.1 | 0.25 | 10 | | | |
| Gefitinib | | | | | | | | |
| Erlotinib | | | | | | | | |
| Sorafenib | potent | potent | potent | | | | potent | potent |
| Sunitinib | 0.01 | 0.01 | 0.01 | 0.8 | | 0.01 | 0.01 | potent |
| Dasatinib | | potent | potent | Potent | | | | |
| Lapatinib | | | | | | | | |

Inhibitory profile (with IC50s when available)

| | | | | | | c- | b- |
|---|---|---|---|---|---|---|---|
| Compound | Flt3 | Src | FGFR | EGFR | HER2 | Raf | Raf |
| Imatinib | | | | | | | |
| Gefitinib | | | | potent | | | |
| Erlotinib | | | | potent | potent | | |
| Sorafenib | potent | | | | | potent | potent |
| Sunitinib | 0.05 | | 0.9 | | | | |
| Dasatinib | | potent | | | | | |
| Lapatinib | | | | potent | potent | | |

TABLE 7
Examples of small molecule tyrosine kinase inhibitors in clinical development, that
represent potential therapeutics for inflammatory and other autoimmune diseases.

| | | | Inhibitory profile (with IC50s when available) | | | | | | | | | |
|---|---|---|---|---|---|---|---|---|---|---|---|---|
| Compound | Other | | c- | | | | VEGFR1 | VEGFR2 | VEGFR3 | | | |
| name | names | Company | Fms | PDGFR | c-Kit | Abl | (Flt1) | (KDR) | (Flt4) | Flt3 | Src | EGFR |
| Pazopanib | GW786034 | GlaxoSmith | | potent | potent | | potent | potent | potent | | | |
| | | Kline | | | | | | | | | | |
| Vatalanib | PTK787/ZK | Novartis | 1.2 | 0.2 | 0.4 | | potent | 0.3 | potent | | | |
| | 222584 | | | | | | | | | | | |
| Vandetanib | ZD6474, | AstraZeneca | | | | | | potent | | | | potent |
| | Zactima | | | | | | | | | | | |
| Cediranib | AZD2171 | AstraZeneca | | potent? | potent? | | potent | potent | potent | | | |
| Semaxanib | SU5416 | Pfizer | | | 0.03 | | 1 | 1 | | 0.25 | | |
| Axitinib | AG-013736 | Pfizer | potent | potent | potent | potent | potent | potent | potent | | | |
| AMG 706 | | Amgen | | potent | potent | | potent | potent | potent | | | |
| Nilotinib | AMN107 | Novartis | | 0.001 | 0.03 | 0.001 | | | | | | |
| CP-690 | | Pfizer | | | | | | | | | | |
| Lestaurtinib | CEP-701 | Cephalon | | | | | | | | potent | | |
| PKC412 | CGP41251 | Novartis | | potent | 0.03 | | | potent | | potent | | |
| AZD0530 | | AstraZeneca | | | | potent | | | | | potent | |
| Tandutinib | MLN518 | Millennium | | 0.2 | 0.2 | 0.1 | 0.2 | | | potent | | |
| | | Pharma. | | | | | | | | | | |
| EKB-569 | | Wyeth | | | | | | | | | | potent |
| SKI-606 | | Wyeth | | | | potent | | | | | potent | |
| PKI-166 | CGP75166 | Novartis | | | | | | | | | | potent |
| CHIR258 | | Novartis | 0.03 | | | 3 | | 0.05 | | | | |
| SU6668 | | Pfizer | | 0.01 | 0.3 | | | 2 | | | | 1 |

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

It is Claimed:

1. A method for treating an inflammatory disease, comprising:
orally administering a tyrosine kinase inhibitor to a subject suffering from an inflammatory disease in an amount sufficient to inhibit the activity of at least one receptor tyrosine kinase.

2. The method of claim 1, wherein said tyrosine kinase inhibitor is selected from imatinib, CGP53716, SU9518, PD166326, and GW2580.

3. The method of claim 2, wherein the tyrosine kinase inhibitor is imatinib and the receptor tyrosine kinase is selected from c-Fms, c-Kit, PDGFRα, PDGFRβ, FGFR and Abl.

4. The method of claim 2, wherein the tyrosine kinase inhibitor is CGP53716 and the receptor tyrosine kinase is selected from PDGFR, FGFR and c-Kit.

5. The method of claim 2, wherein the tyrosine kinase inhibitor is GW2580 and the receptor tyrosine kinase is selected from c-Fms and PDGFR.

6. The method of claim 2, wherein the tyrosine kinase inhibitor is PD166326 and the receptor tyrosine kinase is selected from c-Kit and Abl.

7. The method of claim 2, wherein the tyrosine kinase inhibitor is SU9518 and the receptor tyrosine kinase is PDGFR and FGFR.

8. The method of claim 1, wherein said inflammatory disease is an autoimmune disease.

9. The method of claim 8, wherein said inflammatory disease is rheumatoid arthritis.

10. The method of claim 8, wherein said inflammatory disease is systemic sclerosis.

11. The method of claim 8, wherein said inflammatory disease is multiple sclerosis.

12. The method of claim 8, wherein said inflammatory disease is selected from, psoriasis, psoriatic arthritis, Crohn's disease, systemic lupus erythematosus, and pulmonary fibrosis.

13. The method of claim 1, wherein said tyrosine kinase inhibitor is orally administered at a dose that achieves blood levels of about 0.2 micromolar.

14. The method of claim 1, wherein said tyrosine kinase inhibitor is orally administered at a dose that achieves blood levels of about 1 micromolar.

15. The method of claim 1, wherein said tyrosine kinase inhibitor is orally administered at a dose that achieves blood levels of about 5 micromolar.

16. The method of claim 1, wherein said tyrosine kinase inhibitor is orally administered about once per day.

17. A method for treating an inflammatory disease, comprising orally administering a tyrosine kinase inhibitor to a subject suffering from an inflammatory disease in an amount sufficient to inhibit two or more kinases to treat the inflammatory disease.

18. The method of claim 17, wherein said tyrosine kinase inhibitor is a single compound.

19. The method of claim 17, wherein the tyrosine kinase inhibitor inhibits PDGFR.

20. The method of claim 17, wherein the tyrosine kinase inhibitor inhibits c-Kit.

21. The method of claim 17, wherein the tyrosine kinase inhibitor inhibits c-Fms.

22. The method of claim 17, wherein the tyrosine kinase inhibitor inhibits c-Abl.

23. The method of claim 17, wherein the tyrosine kinase inhibitor inhibits FGFR.

24. The method of claim 17, wherein said inflammatory disease is an autoimmune disease.

25. The method of claim 24, wherein said inflammatory disease is rheumatoid arthritis.

26. The method of claim 24, wherein said inflammatory disease is systemic sclerosis.

27. The method of claim 24, wherein said inflammatory disease is multiple sclerosis.

28. The method of claim 24, wherein said inflammatory disease is selected from, psoriasis, psoriatic arthritis, Crohn's disease, systemic lupus erythematosus, and pulmonary fibrosis.

29. The method of claim 17, wherein said tyrosine kinase inhibitor is orally administered at a dose that achieves blood levels of about 0.2 micromolar.

30. The method of claim 17, wherein said tyrosine kinase inhibitor is orally administered at a dose that achieves blood levels of about 1 micromolar

31. The method of claim 17, wherein said tyrosine kinase inhibitor is orally administered at a dose that achieves blood concentrations of about 5 micromolar.

32. The method of claim 17, wherein said tyrosine kinase inhibitor is orally administered about once per day.

33. The method of claim 17, wherein said tyrosine kinase inhibitor is selected from imatinib, CGP53716, SU9518, PD166326, and GW2580.
